(19) United States
(12) Patent Application Publication
(10) Pub. No.: US 2026/0274940 A1
(43) Pub. Date: Sep. 17, 2026

(54) METHODS TO TREAT CANCER USING ANTI-IL-25 ANTIBODY

(71) Applicant: The Trustees of Columbia University in the City of New York, New York, NY (US)

(72) Inventors: Adam MOR, Old Tappan, NJ (US); Xizi HU, New York, NY (US)

(21) Appl. No.: 19/472,473

(22) PCT Filed: Apr. 5, 2024
(86) PCT No.: PCT/US2024/023452

§ 371 Date: Oct. 3, 2025

Related U.S. Application Data
(60) Provisional application No. 63/494,433, filed on Apr. 5, 2023.
(60) Provisional application No. 63/494,605, filed on Apr. 6, 2023.
(60) Provisional application No. 63/496,004, filed on Apr. 13, 2023.
(60) Provisional application No. 63/496,005, filed on Apr. 13, 2023.
(60) Provisional application No. 63/599,390, filed on Nov. 15, 2023.

(51) Int. Cl. C07K 16/24; A61P 35/00

(52) U.S. Cl. CPC C07K 16/244; A61P 35/00

(57) ABSTRACT

The subject matter described here relates to a method for treating cancer in a subject in need thereof comprising administering to the subject a therapeutically effective amount of an anti-IL-25 antibody or antigen binding fragment thereof.

This International Patent application claims the benefit of and priority to U.S. Provisional Application No. 63/494,433, filed Apr. 5, 2023; U.S. Provisional Application No. 63/494,605, filed Apr. 6, 2023; U.S. Provisional Application No. 63/496,004, filed Apr. 13, 2023; U.S. Provisional Application No. 63/496,005, filed Apr. 13, 2023; and U.S. Provisional Application No. 63/599,390, filed Nov. 15, 2023, the contents of each of which are hereby incorporated by reference in their entireties.

### STATEMENT REGARDING FEDERALLY SPONSORED RESEARCH OR DEVELOPMENT

This invention was made with government support under CA231277, AI125640, AI175498, AI150597 and AI013696 awarded by the National Institutes of Health. The government has certain rights in the invention.

This patent disclosure contains material that is subject to copyright protection. The copyright owner has no objection to the facsimile reproduction by anyone of the patent document or the patent disclosure as it appears in the U.S. Patent and Trademark Office patent file or records but otherwise reserves any and all copyright rights.

### INCORPORATION BY REFERENCE

All documents cited herein are incorporated herein by reference in their entireties.

### TECHNICAL FIELD

The present invention relates generally to the treatment or prevention of cancer with anti-IL25 antibodies. More particularly, the present invention relates to anti-IL25 antibodies for cancer treatment.

### BACKGROUND

In 2023, there were 1.9 million new cancer cases in the U.S., leading to over 600,000 deaths. Cancer immunotherapy, using one's immune system to fight cancer, is at the frontier of cancer treatment. Several types of cancer immunotherapy include immune checkpoint inhibitors (ICI), CAR-T cells, engineering TCR, tumor-infiltrating lymphocytes, and therapeutic cancer vaccines. Among the different types of cancer immunotherapy, ICI is the least invasive, most widely applicable, and most cost-effective. However, the current response rate to ICIs is only 12.5%, due to primary and acquired resistance. There is a need for alternative, effective non, non-invasive therapies for cancer treatment. Studies have showed an inverse correlation between cancer patients' survival and the levels of IL-25.

### SUMMARY

In certain aspects, the present disclosure provides a method for treating cancer in a subject in need thereof comprising administering to the subject a therapeutically effective amount of an anti-IL-25 antibody or antigen binding fragment thereof.

In some embodiments, the anti-IL-25 antibody or antigen binding fragment thereof is administered as a monotherapy. In some embodiments, the anti-IL-25 antibody or antigen binding fragment thereof is a monoclonal antibody or antigen binding fragment thereof. In some embodiments, the anti-IL-25 antibody or antigen binding fragment thereof is LNR-125 or antigen binding fragment thereof. In some embodiments, the anti-IL-25 antibody or antigen binding fragment thereof is LNR-125.38 or antigen binding fragment thereof. In some embodiments, the anti-IL-25 antibody or antigen binding fragment thereof is a humanized form of LNR-125 or antigen binding fragment thereof.

In some embodiments, the anti-IL-25 antibody or antigen binding fragment thereof, comprises:
a first arm comprising a first variable heavy chain domain and a first variable light chain domain, wherein a portion of the first arm is capable of binding to a portion of an IL-25; and a second arm comprising a second variable heavy chain domain and a second variable light chain domain, wherein a portion of the second arm is capable of binding to a portion of the IL-25 protein; wherein the first and second arms each further comprise a fragment, crystallizable (Fc) domain.

In some embodiments, the first and second arms each further comprise a CH1 domain, a hinge domain, and a CL domain. In some embodiments, the portion of IL-25 bound by the first arm and second arm is the same.

In some embodiments, the first variable heavy chain domain of the first arm is encoded by a first polypeptide chain; the first variable light chain domain of the first arm is encoded by a second polypeptide chain; the second variable heavy chain domain of the second arm is encoded by a third polypeptide chain; the second variable light chain domain of the second arm is encoded by a fourth polypeptide chain; and the first variable heavy chain domain and first variable light chain domain form a first IL-25 binding site and wherein the second variable heavy chain domain and second variable light chain domain form a second IL-25 binding site.

In some embodiments, the first and second IL-25 binding sites are the same. In some embodiments, the first and third polypeptide chain each further encode a hinge domain, a CH1 domain, and the Fc domain, and wherein the second and fourth polypeptide chain each further encode a CL domain. In some embodiments, the first and third polypeptide chains comprise the same sequence and the second and fourth polypeptide chains comprise the same sequence.

In some embodiments, the first and second variable heavy chain domain each comprises HCDR1 comprising SEQ ID NO: 1, HCDR2 comprising SEQ ID NO: 2, and HCDR3 comprising SEQ ID NO: 3 and wherein the first and second variable light chain domain each comprises LCDR1 comprising SEQ ID NO: 4, LCDR2 comprising SEQ ID NO: 5, and LCDR3 comprising SEQ ID NO: 6.

In some embodiments, the first and second variable heavy chain domain each comprises HCDR1 comprising SEQ ID NO: 9, HCDR2 comprising SEQ ID NO: 10, and HCDR3 comprising SEQ ID NO: 11 and the first and second variable light chain domain each comprises LCDR1 comprising SEQ ID NO: 12, LCDR2 comprising SEQ ID NO: 13, and LCDR3 comprising SEQ ID NO: 14.

In some embodiments, the first and second variable heavy chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 7 and wherein the first and second variable light chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 8.

In some embodiments, the first and second variable heavy chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 17 and wherein the first and second variable light chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 18.

In some embodiments, the first and second variable heavy chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 15 and wherein the first and second variable light chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 16.

In some embodiments, the first and second variable heavy chain domain each comprises an amino acid sequence of SEQ ID NO: 7 and wherein the first and second variable light chain domain each comprises an amino acid sequence of SEQ ID NO: 8, or wherein the first and second variable heavy chain domain each comprises an amino acid sequence of SEQ ID NO: 15 and the first and second variable light chain domain each comprises an amino acid sequence of SEQ ID NO: 16.

In some embodiments, the first and third polypeptide chain each comprises an amino acid sequence comprising SEQ ID NO: 7 and the second and fourth polypeptide chain each comprises an amino acid sequence comprising SEQ ID NO: 8 or wherein the first and third polypeptide chain each comprises an amino acid sequence comprising SEQ ID NO: 15 and the second and fourth polypeptide chain each comprises an amino acid sequence comprising SEQ ID NO: 16.

In some embodiments, the first and second polypeptide chains are linked by one or more covalent disulfide bonds and the third and fourth polypeptide chains are linked by one or more covalent disulfide bonds. In some embodiments, the first and third polypeptide chains are linked by one or more covalent disulfide bonds.

In some embodiments, the first and second variable heavy chain domain each comprises an amino acid sequence of SEQ ID NO: 17, wherein the first and second variable light chain domain each comprises an amino acid sequence of SEQ ID NO: 18.

In some embodiments, the first and third polypeptide chain each comprises an amino acid sequence comprising SEQ ID NO: 17 and the second and fourth polypeptide chain each comprises an amino acid sequence comprising SEQ ID NO: 18.

In some embodiments, the first and second polypeptide chains are linked by one or more covalent disulfide bonds and the third and fourth polypeptide chains are linked by one or more covalent disulfide bonds.

In some embodiments, the first and third polypeptide chains are linked by one or more covalent disulfide bonds.

In some embodiments, the subject has a solid tumor. In some embodiments, the cancer is lung carcinoma, breast cancer, hepatoma, Carcinoma of prostate gland, renal cell carcinoma, hepatocellular carcinoma, prostate cancer, melanoma, poeciliopsis lucida hepatocellular carcinoma, squamous cell carcinoma, bladder transitional cell carcinoma, glioma, myeloid leukemia, neuroblastoma tumor, stage IV breast cancer, mammary carcinoma cell line, renal cortical adenocarcinoma, B cell lymphoma, lymphoma, plasmacytoma, pancreatic cancer, or acute myeloid leukemia. In some embodiments, the cancer is breast cancer. In some embodiments, the cancer is melanoma. In some embodiments, the cancer is ovarian cancer. In some embodiments, the subject has a hematopoietic malignancy.

In some embodiments, the tumor of the subject is reduced in volume. In some embodiments, growth of a tumor or cancer cells of the subject is inhibited.

In some embodiments, the subject is a human.

### BRIEF DESCRIPTION OF FIGURES

The patent or application file contains at least one drawing originally in color. To conform to the requirements for PCT patent applications, many of the figures presented herein are black and white representations of images originally created in color.

FIGS. 1A-C show anti-IL25 tumor growth inhibition experiment conducted on a tumor cell line, EO771 (medullary breast adenocarcinoma). FIG. 1A shows a schematic overview of anti-IL-25 mono-treatment experiment in B6 mice inoculated with EO771 (medullary breast adenocarcinoma) tumor cells. FIG. 1B shows tumor growth curves and. FIG. 1C shows quantification for Day 18 tumor volume (left hand bar is untreated, right hand bar is treated). EO771 tumors grow slower in mice treated with anti-IL25 than in untreated mice.

### DETAILED DESCRIPTION

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

The term “therapeutically effective amount,” as used herein, refers to an amount or a concentration of one or more compounds or a pharmaceutical composition described herein utilized for a period of time (including in vitro and in vivo acute or chronic administration and periodic or continuous administration) that is effective within the context of its administration for causing an intended effect or physiological outcome.

As used herein, the term “subject” refers to a vertebrate animal. In one embodiment, the subject is a mammal or a mammalian species. In one embodiment, the subject is a human. In one embodiment, the subject is a healthy human adult. In other embodiments, the subject is a non-human vertebrate animal, including, without limitation, non-human primates, laboratory animals, livestock, racehorses, domesticated animals, and non-domesticated animals. In one embodiment, the term “human subjects” means a population of healthy human adults.

All patent applications, published patent applications, issued and granted patents, texts, and literature references cited in this specification are hereby incorporated herein by reference in their entirety to more fully describe the state of the art to which the present disclosed subject matter pertains.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Described herein are methods of treating cancer (e.g., breast cancer). Specifically, described herein are IL-25 antagonists (e.g., anti-IL-25 antibodies, siIL-25). Further described herein are methods of treating cancer (e.g., breast cancer) using IL-25 antagonists (e.g., anti-IL-25 antibodies, siIL-25).

As used herein, “IL-25” refers to Interleukin-25. The imbalance of immune cell-derived factors such as cytokines is among the several mechanisms that play a central role in cancer progression. IL-25, also known as IL-17E, is a cytokine that belongs to the IL-17 cytokine family and is secreted by type 2 helper T cells (Th2) and mast cells. IL-25 induces the production of other cytokines, including IL-4, IL-5, and IL-13, in multiple tissues and stimulates the expansion of eosinophils. IL-25 exerts both a tumor-suppressive and tumor-supportive role. IL-25 exerts a tumor-suppressive role through inducing infiltration of eosinophils and B cells into the tumor microenvironment and activating the apoptotic pathways. Hence anti-IL-25 antibodies have been advanced as a potential complementary approach to the other anticancer agent. However, there is still a need for effective antagonists of IL-25 that are useful in the treatment of cancers, in particular colon cancer and breast cancer.

### Methods of Treatment

In certain aspects, the present disclosure provides a method for treating cancer in a subject in need thereof comprising administering to the subject a therapeutically effective amount of an anti-IL-25 antibody or antigen binding fragment thereof.

In some embodiments, the anti-IL-25 antibody or antigen binding fragment thereof is administered as a monotherapy. In some embodiments, the anti-IL-25 antibody or antigen binding fragment thereof is a monoclonal antibody or antigen binding fragment thereof. In some embodiments, the anti-IL-25 antibody or antigen binding fragment thereof is LNR-125 or antigen binding fragment thereof. In some embodiments, the anti-IL-25 antibody or antigen binding fragment thereof is LNR-125.38 or antigen binding fragment thereof. In some embodiments, the anti-IL-25 antibody or antigen binding fragment thereof is a humanized form of LNR-125 or antigen binding fragment thereof.

In some embodiments, the anti-IL-25 antibody or antigen binding fragment thereof, comprises: a first arm comprising a first variable heavy chain domain and a first variable light chain domain, wherein a portion of the first arm is capable of binding to a portion of an IL-25; and a second arm comprising a second variable heavy chain domain and a second variable light chain domain, wherein a portion of the second arm is capable of binding to a portion of the IL-25 protein; wherein the first and second arms each further comprise a fragment, crystallizable (Fc) domain.

In some embodiments, the first and second arms each further comprise a CH1 domain, a hinge domain, and a CL domain. In some embodiments, the portion of IL-25 bound by the first arm and second arm is the same.

In some embodiments, the first variable heavy chain domain of the first arm is encoded by a first polypeptide chain; the first variable light chain domain of the first arm is encoded by a second polypeptide chain; the second variable heavy chain domain of the second arm is encoded by a third polypeptide chain; the second variable light chain domain of the second arm is encoded by a fourth polypeptide chain; and the first variable heavy chain domain and first variable light chain domain form a first IL-25 binding site and wherein the second variable heavy chain domain and second variable light chain domain form a second IL-25 binding site.

In some embodiments, the first and second IL-25 binding sites are the same. In some embodiments, the first and third polypeptide chain each further encode a hinge domain, a CH1 domain, and the Fc domain, and wherein the second and fourth polypeptide chain each further encode a CL domain. In some embodiments, the first and third polypeptide chains comprise the same sequence and the second and fourth polypeptide chains comprise the same sequence.

In some embodiments, the first and second variable heavy chain domain each comprises HCDR1 comprising SEQ ID NO: 1, HCDR2 comprising SEQ ID NO: 2, and HCDR3 comprising SEQ ID NO: 3 and wherein the first and second variable light chain domain each comprises LCDR1 comprising SEQ ID NO: 4, LCDR2 comprising SEQ ID NO: 5, and LCDR3 comprising SEQ ID NO: 6.

In some embodiments, the first and second variable heavy chain domain each comprises HCDR1 comprising SEQ ID NO: 9, HCDR2 comprising SEQ ID NO: 10, and HCDR3 comprising SEQ ID NO: 11 and the first and second variable light chain domain each comprises LCDR1 comprising SEQ ID NO: 12, LCDR2 comprising SEQ ID NO: 13, and LCDR3 comprising SEQ ID NO: 14.

In some embodiments, the first and second variable heavy chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 7 and wherein the first and second variable light chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 8.

In some embodiments, the first and second variable heavy chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 17 and wherein the first and second variable light chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 18.

In some embodiments, the first and second variable heavy chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 15 and wherein the first and second variable light chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 16.

In some embodiments, the first and second variable heavy chain domain each comprises an amino acid sequence of SEQ ID NO: 7 and wherein the first and second variable light chain domain each comprises an amino acid sequence of SEQ ID NO: 8, or wherein the first and second variable heavy chain domain each comprises an amino acid sequence of SEQ ID NO: 15 and the first and second variable light chain domain each comprises an amino acid sequence of SEQ ID NO: 16.

In some embodiments, the first and third polypeptide chain each comprises an amino acid sequence comprising SEQ ID NO: 7 and the second and fourth polypeptide chain each comprises an amino acid sequence comprising SEQ ID NO: 8 or wherein the first and third polypeptide chain each comprises an amino acid sequence comprising SEQ ID NO: 15 and the second and fourth polypeptide chain each comprises an amino acid sequence comprising SEQ ID NO: 16.

In some embodiments, the first and second polypeptide chains are linked by one or more covalent disulfide bonds and the third and fourth polypeptide chains are linked by one or more covalent disulfide bonds. In some embodiments, the first and third polypeptide chains are linked by one or more covalent disulfide bonds.

In some embodiments, the first and second variable heavy chain domain each comprises an amino acid sequence of SEQ ID NO: 17, wherein the first and second variable light chain domain each comprises an amino acid sequence of SEQ ID NO: 18.

In some embodiments, the first and third polypeptide chain each comprises an amino acid sequence comprising SEQ ID NO: 17 and the second and fourth polypeptide chain each comprises an amino acid sequence comprising SEQ ID NO: 18.

In some embodiments, the first and second polypeptide chains are linked by one or more covalent disulfide bonds and the third and fourth polypeptide chains are linked by one or more covalent disulfide bonds.

In some embodiments, the first and third polypeptide chains are linked by one or more covalent disulfide bonds.

In some embodiments, the IL-25 antagonist comprises several genome editing techniques such as RNAi (RNA interference), zinc finger nucleases (ZFNs), a TALE-effector domain nuclease (TALLEN), prime editing and base editing, CRISPR/Cas9 systems which are known in the art. In some embodiment, the CRISPR/Cas9 systems comprise a guide RNA (gRNA) or a single-molecule guide RNA (sgRNA). In some embodiment, the gRNA or sgRNA comprises a spacer sequence that is complementary to a portion of a nucleic acid sequence encoding IL-25. In some embodiments, the IL-25 antagonist is an antisense RNA that specifically targets IL-25, or a small molecule IL-25 antagonist.

In certain aspects, the present disclosure provides a method for treating cancer in a subject in need thereof comprising administering to the subject a composition comprising a therapeutically effective amount of an anti-IL-25 antagonist. In some embodiments, the IL-25 antagonist is an IL-25 small interfering ribonucleic acid (siIL-25). In some embodiments, the siIL-25 comprises the nucleic acid sequence of any of the siRNA sequences disclosed in Table 1. In some embodiments, the IL-25 antagonist is an IL-25 short-hairpin ribonucleic acid (shIL-25).

In some embodiments, the shIL-25 comprises the nucleic acid sequence of any of the siIL-25 sequences disclosed in Table 1, of SEQ ID NO:9 (ctagtgtagttactagtcttttgaca), or of SEQ ID NO:10 (atttgtttgtttactcatcactcag). In some embodiments, the composition comprises a viral vector comprising a nucleic acid sequence encoding a shIL-25. In some embodiments, the viral vector is an adeno-associated vector (AAV).

In various embodiments, the present application discloses a composition comprising IL-25 siRNA. In various embodiments, the siRNA is at least 75%, 80%, 85%, 90%, 95%, 96%, 97%, 98% or 99% identical to the siRNA designs listed in Table 1, to SEQ ID NO: 9 (ctagtgtagttactagtcttttgaca), or to SEQ ID NO:10 (atttgtttgtttactcatcactcag). In some embodiments, the siRNA consists of a siRNA nucleic acid sequence of Table 1, of SEQ ID NO: 9 (ctagtgtagttactagtcttttgaca), or of SEQ ID NO:10 (atttgtttgtttactcatcactcag). In various embodiments, the present application discloses a composition comprising IL-25 shRNA. For example, in various embodiments the composition is a vector encoding a shRNA wherein the shRNA comprises a nucleic acid sequence encoding the nucleic acid sequences provided in Table 1, SEQ ID NO:9 (ctagtgtagttactagtcttttgaca), or SEQ ID NO:10 (atttgtttgtttactcatcactcag). In various embodiments, the shRNA comprises a nucleic acid sequence at least 75%, 80%, 85%, 90%, 95%, 96%, 97%, 98% or 99% identical to the nucleic acid sequence as provided in Table 1. In some embodiments, the shRNA consists of a nucleic acid sequence of Table 1. In various embodiments, the vector is a viral vector comprising a nucleic acid encoding a IL-25 short-hairpin RNA (shRNA). In various embodiments, the viral vector is an AAV vector. In various embodiments, the viral vector is a vector that preferentially targets the liver or liver cells. In various embodiments, the AAV is AAV 1, 2, 3, 4, 5, 6, 7, 8, 9, or 10 or variants thereof. In various embodiments, the AAV is AAV8 or a variant thereof. In some embodiments, the AAV, including the AAV8, is a hepatocyte-targeted AAV. In some embodiments, the composition comprises hepatocyte-targeted AAV8 comprising a nucleic acid encoding IL-25 short-hairpin RNA (shRNA).

In some embodiments, the subject has a solid tumor. In some embodiments, the cancer is lung carcinoma, breast cancer, hepatoma, Carcinoma of prostate gland, renal cell carcinoma, hepatocellular carcinoma, prostate cancer, melanoma, poeciliopsis lucida hepatocellular carcinoma, squamous cell carcinoma, bladder transitional cell carcinoma, glioma, myeloid leukemia, neuroblastoma tumor, stage IV breast cancer, mammary carcinoma cell line, renal cortical adenocarcinoma, B cell lymphoma, lymphoma, plasmacytoma, pancreatic cancer, or acute myeloid leukemia. In some embodiments, the cancer is breast cancer. In some embodiments, the cancer is melanoma. In some embodiments, the cancer is ovarian cancer. In some embodiments, the subject has a hematopoietic malignancy.

In some embodiments, the tumor of the subject is reduced in volume. In some embodiments, growth of a tumor or cancer cells of the subject is inhibited.

In some embodiments, the subject is a human.

In certain aspects, the subject matter disclosed herein relates to a preventive medical treatment started after following diagnosis of a disease (e.g., cancer) in order to prevent the disease from worsening or curing the disease. In one embodiment, the subject matter disclosed herein relates to prophylaxis of subjects who are believed to be at risk for moderate or severe disease associated with cancer or have previously been diagnosed with another disease, such as cancer. In one embodiment, the subjects can be administered the pharmaceutical composition described herein. The invention contemplates using any of the antibodies produced by the systems and methods described herein. In one embodiment, the compositions described herein can be administered subcutaneously via syringe or any other suitable method know in the art.

The compound(s) or combination of compounds disclosed herein, or pharmaceutical compositions may be administered to a cell, mammal, or human by any suitable means. Non-limiting examples of methods of administration include, among others, (a) administration though oral pathways, which includes administration in capsule, tablet, granule, spray, syrup, or other such forms; (b) administration through non-oral pathways such as intraocular, intranasal, intraauricular, rectal, vaginal, intraurethral, transmucosal, buccal, or transdermal, which includes administration as an aqueous suspension, an oily preparation or the like or as a drip, spray, suppository, salve, ointment or the like; (c) administration via injection, including subcutaneously, intraperitoneally, intravenously, intramuscularly, intradermally, intraorbitally, intracapsularly, intraspinally, intrasternally, or the like, including infusion pump delivery; (d) administration locally such as by injection directly in the renal or cardiac area, e.g., by depot implantation; (e) administration topically; as deemed appropriate by those of skill in the art for bringing the compound or combination of compounds disclosed herein into contact with living tissue; (f) administration via inhalation, including through aerosolized, nebulized, and powdered formulations; (g) administration through implantation; and administration via electroporation.

In some embodiments, one or more antibodies disclosed herein are prepared in a cocktail of DNA-encoding antibodies or mRNA-encoding antibodies and delivered by electroporation to a subject for in vivo expression of the encoded antibodies.

As will be readily apparent to one skilled in the art, the effective in vivo dose to be administered and the particular mode of administration will vary depending upon the age, weight and species treated, and the specific use for which the compound or combination of compounds disclosed herein are employed. The determination of effective dose levels, that is the dose levels necessary to achieve the desired result, can be accomplished by one skilled in the art using routine pharmacological methods. Typically, human clinical applications of products are commenced at lower dose levels, with dose level being increased until the desired effect is achieved. Alternatively, acceptable in vitro studies can be used to establish useful doses and routes of administration of the compositions identified by the present methods using established pharmacological methods. Effective animal doses from in vivo studies can be converted to appropriate human doses using conversion methods known in the art (e.g., see Nair A B, Jacob S. A simple practice guide for dose conversion between animals and human. Journal of basic and clinical pharmacy. 2016 March; 7(2):27.)

As described herein, the methods of treatment refer generally to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. Methods described herein covers any treatment of a disease in a subject, and includes: (a) preventing the disease or symptom from occurring in a subject which may be predisposed to the disease or symptom, may or may not be diagnosed as having it; (b) inhibiting the disease symptom, i.e., arresting its development; or (c) relieving the disease symptom, i.e., causing regression of the disease or symptom.

A therapeutically effective amount of an agent or composition disclosed herein, for example, is one that is effective for preventing, ameliorating, treating or delaying the onset of a disease or condition.

Pharmaceutical compositions can be administered to any animal that can experience the beneficial effects of the agents of the invention. Such animals include humans and non-humans such as primates, pets and farm animals.

The present invention also comprises pharmaceutical compositions comprising the therapeutic agents described herein. Routes of administration and dosages of effective amounts of the pharmaceutical compositions comprising the agents are also disclosed. The agents of the present invention can be administered in combination with other pharmaceutical agents in a variety of protocols for effective treatment of disease.

Pharmaceutical compositions of the present invention are administered to a subject in a manner known in the art. The dosage administered will be dependent upon the age, health, and weight of the recipient, kind of concurrent treatment, if any, frequency of treatment, and the nature of the effect desired. One may administer the pharmaceutical compositions in a local rather than systemic manner, for example, via injection of directly into the desired target site, often in a depot or sustained release formulation. Furthermore, one may administer the composition in a targeted drug delivery system.

One of ordinary skill in the art will appreciate that a method of administering pharmaceutically effective amounts of pharmaceutical compositions to a patient in need thereof, can be determined empirically, or by standards currently recognized in the medical arts. The agents can be administered to a patient as pharmaceutical compositions in combination with one or more pharmaceutically acceptable excipients. It will be understood that, when administered to a human patient, the total daily usage of the agents of the pharmaceutical compositions of the present invention will be decided within the scope of sound medical judgment by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, gender and diet of the patient; the time of administration, route of administration, and rate of excretion of the agent; the duration of the treatment; drugs used in combination or coincidental with the specific agent; and like factors well known in the medical arts. It is well within the skill of the art to start doses of the agents at levels lower than those required to achieve the desired therapeutic effect and to gradually increase the dosages until the desired effect is achieved.

The practice of aspects of the present invention can employ, unless otherwise indicated, conventional techniques of cell biology, cell culture, molecular biology, transgenic biology, microbiology, recombinant DNA, and immunology, which are within the skill of the art. Such techniques are explained fully in the literature. See, e.g., Molecular Cloning A Laboratory Manual, 3rd Ed., ed. by Sambrook (2001), Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription and Translation (B. D. Hames & S. J. Higgins eds. 1984); Culture Of Animal Cells (R. I. Freshney, Alan R. Liss, Inc., 1987); Immobilized Cells and Enzymes (IRL Press, 1986); B. Perbal, A Practical Guide To Molecular Cloning (1984); the series, Methods In Enzymology (Academic Press, Inc., N.Y.), specifically, Methods In Enzymology, Vols. 154 and 155 (Wu et al. eds.); Gene Transfer Vectors For Mammalian Cells (J. H. Miller and M. P. Calos eds., 1987, Cold Spring Harbor Laboratory); Immunochemical Methods In Cell And Molecular Biology (Caner and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986) and subsequent versions thereof. All patents, patent applications and references cited herein are incorporated by reference in their entireties.

One skilled in the art can obtain a protein in several ways, which include, but are not limited to, isolating the protein via biochemical means or expressing a nucleotide sequence encoding the protein of interest by genetic engineering methods.

### Compositions

In certain aspects, the present disclosure provides a composition comprising a therapeutically effective amounts of anti-IL-25 antibody or antigen binding fragment thereof.

In some embodiments, the composition further comprises one or more pharmaceutically acceptable excipients. In some embodiments, the composition further comprises a package insert or label providing directions for administering the composition.

In some embodiments, the anti-IL-25 antibody or antigen binding fragment thereof is LNR-125 or antigen binding fragment thereof. In some embodiments, the anti-IL-25 antibody or antigen binding fragment thereof is LNR-125.38 or antigen binding fragment thereof. In some embodiments, the anti-IL-25 antibody or antigen binding fragment thereof is a humanized form of LNR-125 or antigen binding fragment thereof.

In some embodiments, the anti-IL-25 antibody or antigen binding fragment thereof, comprises: a first arm comprising a first variable heavy chain domain and a first variable light chain domain, wherein a portion of the first arm is capable of binding to a portion of an IL-25; and a second arm comprising a second variable heavy chain domain and a second variable light chain domain, wherein a portion of the second arm is capable of binding to a portion of the IL-25 protein; wherein the first and second arms each further comprise a fragment, crystallizable (Fc) domain.

In some embodiments, the first and second arms each further comprise a CH1 domain, a hinge domain, and a CL domain. In some embodiments, the portion of IL-25 bound by the first arm and second arm is the same.

In some embodiments, the first variable heavy chain domain of the first arm is encoded by a first polypeptide chain; the first variable light chain domain of the first arm is encoded by a second polypeptide chain; the second variable heavy chain domain of the second arm is encoded by a third polypeptide chain; the second variable light chain domain of the second arm is encoded by a fourth polypeptide chain; and the first variable heavy chain domain and first variable light chain domain form a first IL-25 binding site and wherein the second variable heavy chain domain and second variable light chain domain form a second IL-25 binding site.

In some embodiments, the first and second IL-25 binding sites are the same. In some embodiments, the first and third polypeptide chain each further encode a hinge domain, a CH1 domain, and the Fc domain, and wherein the second and fourth polypeptide chain each further encode a CL domain. In some embodiments, the first and third polypeptide chains comprise the same sequence and the second and fourth polypeptide chains comprise the same sequence.

In some embodiments, the first and second variable heavy chain domain each comprises HCDR1 comprising SEQ ID NO: 1, HCDR2 comprising SEQ ID NO: 2, and HCDR3 comprising SEQ ID NO: 3 and wherein the first and second variable light chain domain each comprises LCDR1 comprising SEQ ID NO: 4, LCDR2 comprising SEQ ID NO: 5, and LCDR3 comprising SEQ ID NO: 6.

In some embodiments, first and second variable heavy chain domain each comprises HCDR1 comprising SEQ ID NO: 9, HCDR2 comprising SEQ ID NO: 10, and HCDR3 comprising SEQ ID NO: 11 and the first and second variable light chain domain each comprises LCDR1 comprising SEQ ID NO: 12, LCDR2 comprising SEQ ID NO: 13, and LCDR3 comprising SEQ ID NO: 14.

In some embodiments, the first and second variable heavy chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 7 and wherein the first and second variable light chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 8.

In some embodiments, the first and second variable heavy chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 17 and wherein the first and second variable light chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 18.

In some embodiments, the first and second variable heavy chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 15 and wherein the first and second variable light chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 16.

In some embodiments, the first and second variable heavy chain domain each comprises an amino acid sequence of SEQ ID NO: 7 and wherein the first and second variable light chain domain each comprises an amino acid sequence of SEQ ID NO: 8, or wherein the first and second variable heavy chain domain each comprises an amino acid sequence of SEQ ID NO: 15 and the first and second variable light chain domain each comprises an amino acid sequence of SEQ ID NO: 16.

In some embodiments, the first and third polypeptide chain each comprises an amino acid sequence comprising SEQ ID NO: 7 and the second and fourth polypeptide chain each comprises an amino acid sequence comprising SEQ ID NO: 8 or wherein the first and third polypeptide chain each comprises an amino acid sequence comprising SEQ ID NO: 15 and the second and fourth polypeptide chain each comprises an amino acid sequence comprising SEQ ID NO: 16.

In some embodiments, the first and second polypeptide chains are linked by one or more covalent disulfide bonds and the third and fourth polypeptide chains are linked by one or more covalent disulfide bonds. In some embodiments, the first and third polypeptide chains are linked by one or more covalent disulfide bonds.

In some embodiments, the first and second variable heavy chain domain each comprises an amino acid sequence of SEQ ID NO: 17, wherein the first and second variable light chain domain each comprises an amino acid sequence of SEQ ID NO: 18.

In some embodiments, the first and third polypeptide chain each comprises an amino acid sequence comprising SEQ ID NO: 17 and the second and fourth polypeptide chain each comprises an amino acid sequence comprising SEQ ID NO: 18.

In some embodiments, the first and second polypeptide chains are linked by one or more covalent disulfide bonds and the third and fourth polypeptide chains are linked by one or more covalent disulfide bonds. In some embodiments, the first and third polypeptide chains are linked by one or more covalent disulfide bonds.

In some, embodiments, the cancer is lung carcinoma, breast cancer, hepatoma, Carcinoma of prostate gland, renal cell carcinoma, hepatocellular carcinoma, prostate cancer, melanoma, poeciliopsis lucida hepatocellular carcinoma, squamous cell carcinoma, bladder transitional cell carcinoma, glioma, myeloid leukemia, neuroblastoma tumor, stage IV breast cancer, mammary carcinoma cell line, renal cortical adenocarcinoma, B cell lymphoma, lymphoma, plasmacytoma, pancreatic cancer, or acute myeloid leukemia.

One of ordinary skill in the art will appreciate that a method of administering pharmaceutically effective amounts of the pharmaceutical compositions of the invention to a patient in need thereof, can be determined empirically, or by standards currently recognized in the medical arts. The agents can be administered to a patient as pharmaceutical compositions in combination with one or more pharmaceutically acceptable excipients. It will be understood that, when administered to a human patient, the total daily usage of the agents of the pharmaceutical compositions of the present invention will be decided within the scope of sound medical judgment by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, gender and diet of the patient; the time of administration, route of administration, and rate of excretion of the agent; the duration of the treatment; drugs used in combination or coincidental with the specific agent; and like factors well known in the medical arts. It is well within the skill of the art to start doses of the agents at levels lower than those required to achieve the desired therapeutic effect and to gradually increase the dosages until the desired effect is achieved.

Dosaging can also be administered in a patient-specific manner to provide a predetermined concentration of the agents in the blood, as determined by techniques accepted and routine in the art.

In some embodiments, the IL-25 antagonist comprises several genome editing techniques such as RNAi (RNA interference), zinc finger nucleases (ZFNs), a TALE-effector domain nuclease (TALLEN), prime editing and base editing, CRISPR/Cas9 systems which are known in the art. In some embodiment, the CRISPR/Cas9 systems comprise a guide RNA (gRNA) or a single-molecule guide RNA (sgRNA). In some embodiment, the gRNA or sgRNA comprises a spacer sequence that is complementary to a portion of a nucleic acid sequence encoding IL-25. In some embodiments, the IL-25 antagonist is an antisense RNA that specifically targets IL-25, or a small molecule IL-25 antagonist.

In certain aspects, the present disclosure provides a method for treating cancer in a subject in need thereof comprising administering to the subject a composition comprising a therapeutically effective amount of an anti-IL-25 antagonist. In some embodiments, the IL-25 antagonist is an IL-25 small interfering ribonucleic acid (siIL-25). In some embodiments, the siIL-25 comprises the nucleic acid sequence of any of the siRNA sequences disclosed in Table 1, of SEQ ID NO:9 (ctagtgtagttactagtcttttgaca), or of SEQ ID NO:10 (atttgtttgtttactcatcactcag). In some embodiments, the IL-25 antagonist is an IL-25 short-hairpin ribonucleic acid (shIL-25).

In some embodiments, the shIL-25 comprises the nucleic acid sequence of any of the siIL-25 sequences disclosed in Table 1, of SEQ ID NO:9 (ctagtgtagttactagtcttttgaca), or of SEQ ID NO:10 (atttgtttgtttactcatcactcag). In some embodiments, the composition comprises a viral vector comprising a nucleic acid sequence encoding a shIL-25. In some embodiments, the viral vector is an adeno-associated vector (AAV).

In various embodiments, the present application discloses a composition comprising IL-25 siRNA. In various embodiments, the siRNA is at least 75%, 80%, 85%, 90%, 95%, 96%, 97%, 98% or 99% identical to the siRNA designs listed in Table 1, to SEQ ID NO: 9 (ctagtgtagttactagtcttttgaca), or to SEQ ID NO:10 (atttgtttgtttactcatcactcag). In some embodiments, the siRNA consists of a siRNA nucleic acid sequence of Table 1, of SEQ ID NO: 9 (ctagtgtagttactagtcttttgaca), or of SEQ ID NO:10 (atttgtttgtttactcatcactcag). In various embodiments, the present application discloses a composition comprising IL-25 shRNA. For example, in various embodiments the composition is a vector encoding a shRNA wherein the shRNA comprises a nucleic acid sequence encoding the nucleic acid sequences provided in Table 1, SEQ ID NO:9 (ctagtgtagttactagtcttttgaca), or SEQ ID NO:10 (atttgtttgtttactcatcactcag). In various embodiments, the shRNA comprises a nucleic acid sequence at least 75%, 80%, 85%, 90%, 95%, 96%, 97%, 98% or 99% identical to the nucleic acid sequence as provided in Table 1. In some embodiments, the shRNA consists of a nucleic acid sequence of Table 1. In various embodiments, the vector is a viral vector comprising a nucleic acid encoding a IL-25 short-hairpin RNA (shRNA). In various embodiments, the viral vector is an AAV vector. In various embodiments, the viral vector is a vector that preferentially targets the liver or liver cells. In various embodiments, the AAV is AAV 1, 2, 3, 4, 5, 6, 7, 8, 9, or 10 or variants thereof. In various embodiments, the AAV is AAV8 or a variant thereof. In some embodiments, the AAV, including the AAV8, is a hepatocyte-targeted AAV. In some embodiments, the composition comprises hepatocyte-targeted AAV8 comprising a nucleic acid encoding IL-25 short-hairpin RNA (shRNA).

### Anti-IL-25 Antibodies

There are five classes of human antibodies (i.e., IgA, IgD, IgE, IgG, and IgM) and each have various isotypes (e.g., IgG1, IgG2, IgG3, IgG4, IgA1, and IgA2). In some embodiments, the antibodies disclosed herein belong to the IgG class. IgG can be further divided into four subclasses: IgG1, IgG2, IgG3, and IgG4. Each subclass has a unique profile with respect to antigen binding, immune complex formation, complement activation, triggering of effector cells, half-life, and placental transport. E.g., see Gestur Vidarsson, et al., IgG Subclasses and Allotypes: From Structure to Effector Functions, 5 Frontiers in Immunology 520 (2014), incorporated by reference herein in its entirety. The term “immunoglobulin” (Ig) is used interchangeably with “antibody” herein.

The IgG immunoglobulin molecule consists of four polypeptide chains, two identical light (L) chains and two identical heavy (H) chains. The four chains are joined by disulfide bonds in a “Y” configuration wherein the light chains bracket the heavy chains starting at the mouth of the “Y” and continuing through the variable region to the dual ends of the “Y”. Each L chain is linked to an H chain by one covalent disulfide bond, while the two H chains are linked to each other by one or more disulfide bonds depending on the H chain isotype. Each H and L chain also has regularly spaced intrachain disulfide bridges. Each heavy chain consists of an N-terminal variable domain (VH) and three constant domains (CH1, CH2, CH3), with an additional “hinge region” between CH1 and CH2. Similarly, the light chains consist of an N-terminal variable domain (VL) and a constant domain (CL). The variable domains of the heavy chain and light chain may be referred to as “VH” and “VL”, respectively. These domains are generally the most variable parts of the antibody (relative to other antibodies of the same class) and contain the antigen binding sites. The VL is aligned with the VH and the CL is aligned with the first constant domain of the heavy chain (CH1). The pairing of a VH and VL together forms a single antigen-binding site. The part of the antibody formed by the lower hinge region and the CH2/CH3 domains of the heavy chain is called “Fc” (“fragment crystalline”). See e.g., Basic and Clinical Immunology, 8th Edition, Daniel P. Sties, Abba I. Terr and Tristram G. Parsolw (eds), Appleton & Lange, Norwalk, CT, 1994, page 71 and Chapter 6, incorporated by reference herein in its entirety.

The variability in an antibody sequence is concentrated in three segments called complementarity determining regions (CDRs) (also called hypervariable regions (HVRs)) both in the light-chain and the heavy chain variable domains. The more highly conserved portions of variable domains are called the framework regions (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies. See Kabat et al, Sequences of Immunological Interest, Fifth Edition, National Institute of Health, Bethesda, MD (1991), incorporated by reference in its entirety herein. The constant domains are not involved directly in the binding of antibody to an antigen, but exhibit various effector functions, such as participation of the antibody in antibody-dependent cellular toxicity.

By way of example, CDRs may be defined using the nomenclature described by Kabat et al. (1991, NIH Publication 91-3242, National Technical Information Service, Springfield, Va.), incorporated by reference in its entirety herein. Specifically, residues 31-35 (CDR-H1), 50-65 (CDR-H2), and 95-102 (CDR-H3) in the heavy chain variable region and residues 24-34 (CDR-L1), 50-56 (CDR-L2), and 89-97 (CDR-L3) in the light chain variable region.

The antibodies of the various embodiments disclosed herein can include one or more of synthetic antibodies, monoclonal antibodies, oligoclonal or polyclonal antibodies, multiclonal antibodies, recombinantly produced antibodies, monospecific antibodies, monovalent antibodies, human antibodies, humanized antibodies, chimeric antibodies, CDR-grafted antibodies, primatized antibodies, single-chain Fv-Fcs (scFv-Fc)), bivalent with four scFv (scFv-Fc-scFv), IgG-scFv, IgM, IgA, trispecific, IgG-dAb, CrossMab 2:1 or 2:2, DVD-IgG, IgG(L)-scFv2, DVD-IgG, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG (four-in-one), scFv-KIH, and any other immunologically-reactive/antigen-binding molecules. In some embodiments, the antibody is a monoclonal antibody.

In some embodiments, the monoclonal antibody comprises a first, second, third and fourth chain. In some embodiments, the first and third chains each comprise a VH domain and the second and fourth chains each comprise a VL domain. In some embodiments, the first and third chains each further comprises a CH1 domain, a hinge domain, and a Fc domain. In some embodiments, the second and fourth chains each further comprises a CL domain. The pairing of the VH and VL of the first and second chains together forms a single antigen-binding site specific for an epitope on IL-25 and the pairing of the VH and VL of the third and fourth chains together forms a single antigen-binding site specific for the same epitope. In some embodiments, the first and second chains are linked by one or more covalent disulfide bonds and the third and fourth chains are linked by one or more covalent disulfide bonds. In some embodiments, the first and third chains are linked by one or more disulfide bonds.

However, the antibodies disclosed herein are not limited to full-length IgG like antibodies. Other immunologically reactive/antigen-binding molecules (including but not limited to, single-chain Fv-Fcs (scFv-Fc), bivalent with four scFv (scFv-Fc-scFv), IgG-scFv, IgM, IgA, trispecific, IgG-dAb, CrossMab 2:1 or 2:2, DVD-IgG, IgG(L)-scFv2, DVD-IgG, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG (four-in-one), and scFv-KIH are also contemplated herein and a person of skill in the art can readily synthesize such molecules using the sequences and identified domains of the heavy and light chains of the anti-IL-25 antibodies disclosed here. For example, in some embodiments, the monoclonal antibody comprises a first and second chain that associate together. In some embodiments, the first chain and second chain each comprises an scFv with specificity for an epitope on IL-25 and the first and second chains each further comprise a Fc domain. An scFv comprises a variable heavy domain and variable light chain domain separated by a linker. In some embodiments, the linker is a glycine-serine linker. In some embodiments, the Fc domain of the first chain comprises knob mutations and the Fc domain of the second chain comprise hole mutations, or vice versa. In some embodiments the antibody is a scFv-Fc antibody comprising a first and second chain that associate together, each chain comprising a variable heavy chain (VH) domain, a linker, a variable light chain (VL) domain, and an Fc domain.

For example, in some embodiments, the monoclonal antibody comprises a first and second chain that associate together. In some embodiments, the first chain and second chain each comprise two scFvs with specificity for an epitope on IL-25 and the first and second chains each further comprise a Fc domain. An scFv comprises a variable heavy domain and variable light chain domain separated by a linker. In some embodiments, the linker is a glycine-serine linker. In some embodiments, the Fc domain of the first chain comprises knob mutations and the Fc domain of the second chain comprise hole mutations, or vice versa. In some embodiments the antibody is a scFv-Fc-scFv antibody comprising a first and second chain that associate together, each chain comprising a first variable heavy chain (VH) domain, a first linker, a first variable light chain (VL) domain, an Fc domain, a second variable heavy chain (VH) domain, a second linker, and a second variable light chain (VL) domain.

In some embodiments, the monoclonal antibodies disclosed herein contain various modifications, substitutions, additions, or deletions to the variable or binding regions of one or more arms of an anti-IL-25 antibody disclosed herein. In some embodiments, the monoclonal antibodies disclosed herein may contain substitutions or modifications of the constant region (i.e., the Fc domain). The antibodies disclosed herein may contain one or more additional amino acid residue substitutions, mutations and/or modifications, which result in a compound with preferred characteristics including, but not limited to: altered pharmacokinetics, increased serum half-life, increase binding affinity, reduced binding affinity, reduced immunogenicity, increased production, altered Fc ligand binding, enhanced or reduced ADCC or CDC activity, altered glycosylation and/or disulfide bonds and modified binding specificity.

IL-25 can be antagonized using antibodies specific for IL-25 or antigen binding fragments thereof. In some embodiments the antibody is monoclonal. Non-limiting examples include LNR-125, humanized versions of LNR-125 (also referred to as LNR125.38), and 22C7 (Pfizer). In some embodiments, the anti-IL-25 antibody is a partially or a fully humanized version of LNR-125.

In some embodiments, the anti-IL-25 antibody or antigen binding fragment thereof for use in the methods of treatment disclosed herein comprises an antigen binding site comprising at least one immunoglobulin heavy chain variable domain (VH) which comprises hypervariable regions HCDR1, HCDR2 and HCDR3 and at least one immunoglobulin light chain variable domain (VL) which comprises hypervariable regions LCDR1, LCDR2, and LCDR3.

In some embodiments, the anti-IL-25 antibody for use in the methods of treatment disclosed herein comprises LNR-125 (also referred to as ABM125) or an antigen binding fragment thereof as disclosed in the U.S. Pat. No. 11,492,397, hereby incorporated in its entirety by reference. In some embodiments, the anti-IL-25 antibody or antigen binding fragment thereof comprises an antigen binding site comprising at least one immunoglobulin heavy chain variable domain with hypervariable regions HCDR1, HCDR2 and HCDR3. HCDR1 comprising the amino acid sequence SEQ ID NO: 1 (TSGMGVG) or the amino acid sequence SEQ ID NO: 9 (SYWIE), HCDR2 comprising the amino acid sequence SEQ ID NO: 2 (HIWWDDVKRYNPALKS) or the amino acid sequence SEQ ID NO: 10 (QILPGIGSTNYNEKFKG), and HCDR3 comprising the amino acid sequence SEQ ID NO: 3 (TLPHFFDY) or the amino acid sequence SEQ ID NO: 11 (GYGNYGDY); or HCDR equivalents thereof. In some embodiments, HCDR1 comprises the amino acid sequence SEQ ID NO: 1, HCDR2 comprises the amino acid sequence SEQ ID NO: 2, and HCDR3 comprises the amino acid sequence SEQ ID NO: 3. In some embodiments, HCDR1 comprises the amino acid sequence SEQ ID NO: 9, HCDR2 comprises the amino acid sequence SEQ ID NO: 10, and HCDR3 comprises the amino acid sequence SEQ ID NO: 11. In some embodiments, the anti-IL-25 antibody or antigen binding fragment thereof can also comprise at least one immunoglobulin light chain variable domain which comprises hypervariable regions LCDR1, LCDR2, and LCDR3. LCDR1 comprising the amino acid sequence SEQ ID NO: 4 (SASSSVSYMY) or the amino acid sequence SEQ ID NO: 12 (RASESVDSYGNSFM), LCDR2 comprising the amino acid sequence SEQ ID NO: 5 (RTSNLAS) or the amino acid sequence SEQ ID NO: 13 (RASNLES), and LCDR3 comprising the amino acid sequence SEQ ID NO: 6 (KQYHSYPPTWT) or the amino acid sequence SEQ ID NO: 14 (QQSNEDPLT), or LCDR equivalents thereof. In some embodiments, LCDR1 comprises the amino acid sequence SEQ ID NO: 4, LCDR2 comprises the amino acid sequence SEQ ID NO: 5, and LCDR3 comprises the amino acid sequence SEQ ID NO: 6. In some embodiments, LCDR1 comprises the amino acid sequence SEQ ID NO: 12, LCDR2 comprises the amino acid sequence SEQ ID NO: 13, and LCDR3 comprises the amino acid sequence SEQ ID NO: 14.

In some embodiments, the anti-IL-25 antibody or antigen binding fragment thereof for use in the methods of treatment disclosed herein comprises an antigen binding site comprising at least one immunoglobulin heavy chain variable domain comprising SEQ ID NO: 7 (QVTLKVSGPGILQPSQTLSLTCSFSGFSLNTSGMGVGWIRQPSGKGLEWLAHIWWD DVKRYNPALKSRLTISKDTSGSQVFLKIASVDTADTATYYCARTLPHFFDYWGQGTT LTVSS) or SEQ ID NO: 15 (EVKVVESGADLMKPGASVKISCKATGYTFSSYWIEWVKQRPGHGLEWIGQILPGIG STNYNEKFKGKATFTADTSSNTAYMQLSSLTSEDSAVYYCARGYGNYGDYWGQGT TVTVSS). In some embodiments, the anti-IL-25 antibody or antigen binding fragment thereof for use in the methods of treatment disclosed herein can also comprise at least one immunoglobulin light chain variable domain comprising SEQ ID NO: 8 (DIQMTQSPAIMSASPGEKVTISCSASSSVSYMYWYQQKSGSSPKPWIYRTSNLASGV PARFSGSGSGTSYSLTISSMEAEDAATYYCKQYHSYPPTWTFGGGTKLEIKR) or SED ID NO: 16 (DIVLTQSPASLAVSLGQRATISCRASESVDSYGNSFMHWYQQKPGQPPKLLIYRASN LESGIPARFSGSGSRTDFTLTINPVEADDVATYYCQQSNEDPLTFGAGTKLELKR). In some embodiments, the anti-IL-25 antibody or antigen binding fragment thereof for use in the methods of treatment disclosed herein comprises at least one immunoglobulin heavy chain variable domain comprising SEQ ID NO: 7 and comprises at least one immunoglobulin light chain variable domain comprising SEQ ID NO: 8. In some embodiments, the anti-IL-25 antibody or antigen binding fragment thereof for use in the methods of treatment disclosed herein comprises at least one immunoglobulin heavy chain variable domain comprising SEQ ID NO: 15 and comprises at least one immunoglobulin light chain variable domain comprising SEQ ID NO: 16. In some embodiments, the anti-IL-25 antibody or antigen binding fragment thereof comprises an antigen binding site comprising at least one immunoglobulin heavy chain variable domain comprising an amino acid sequence having 80, 81, 82, 83, 84, 85, 86, 87, 88, 89, 90, 91, 92, 93, 94, 95, 96, 97, 98, or 99% identity to SEQ ID NO: 7 or SEQ ID NO: 15. In some embodiments, the anti-IL-25 antibody or antigen binding fragment thereof comprises an antigen binding site comprising at least one immunoglobulin heavy chain variable domain comprising an amino acid sequence having 80, 81, 82, 83, 84, 85, 86, 87, 88, 89, 90, 91, 92, 93, 94, 95, 96, 97, 98, or 99% identity to SEQ ID NO: 7 or SEQ ID NO: 15 and comprising HCDR1 comprising the amino acid sequence SEQ ID NO: 1 (TSGMGVG) or SEQ ID NO: 9 (SYWIE), HCDR2 comprising the amino acid sequence SEQ ID NO: 2 (HIWWDDVKRYNPALKS) or SEQ ID NO: 10 (QILPGIGSTNYNEKFKG), and HCDR3 comprising the amino acid sequence SEQ ID NO: 3 (TLPHFFDY) or SEQ ID NO: 11 (GYGNYGDY). In some embodiments, the anti-IL-25 antibody or antigen binding fragment thereof comprises an antigen binding site comprising at least one immunoglobulin heavy chain variable domain comprising an amino acid sequence having 80, 81, 82, 83, 84, 85, 86, 87, 88, 89, 90, 91, 92, 93, 94, 95, 96, 97, 98, or 99% identity to SEQ ID NO: 7 and comprising HCDR1 comprising the amino acid sequence SEQ ID NO: 1, HCDR2 comprising the amino acid sequence SEQ ID NO: 2, and HCDR3 having the amino acid sequence SEQ ID NO: 3. In some embodiments, the anti-IL-25 antibody or antigen binding fragment thereof comprises an antigen binding site comprising at least one immunoglobulin heavy chain variable domain comprising an amino acid sequence having 80, 81, 82, 83, 84, 85, 86, 87, 88, 89, 90, 91, 92, 93, 94, 95, 96, 97, 98, or 99% identity to SEQ ID NO: 15 and comprising HCDR1 comprising the amino acid sequence SEQ ID NO: 9, HCDR2 comprising the amino acid sequence SEQ ID NO: 10, and HCDR3 having the amino acid sequence SEQ ID NO: 11. In some embodiments, the anti-IL-25 antibody or antigen binding fragment thereof can also comprise at least one immunoglobulin light chain variable domain comprising an amino acid sequence having 80, 81, 82, 83, 84, 85, 86, 87, 88, 89, 90, 91, 92, 93, 94, 95, 96, 97, 98, or 99% identity to SEQ ID NO: 8 or 16. In some embodiments, the anti-IL-25 antibody or antigen binding fragment thereof can also comprise at least one immunoglobulin light chain variable domain comprising an amino acid sequence having 80, 81, 82, 83, 84, 85, 86, 87, 88, 89, 90, 91, 92, 93, 94, 95, 96, 97, 98, or 99% identity to SEQ ID NO: 8 or 16 and comprising LCDR1 comprising the amino acid sequence SEQ ID NO: 4 (SASSSVSYMY) or SEQ ID NO: 12 (RASESVDSYGNSFM), LCDR2 comprising the amino acid sequence SEQ ID NO: 5 (RTSNLAS) or SEQ ID NO: 13 (RASNLES), and LCDR3 comprising the amino acid sequence SEQ ID NO: 6 (KQYHSYPPTWT) or SEQ ID NO: 14 (QQSNEDPLT). In some embodiments, the anti-IL-25 antibody or antigen binding fragment thereof can also comprise at least one immunoglobulin light chain variable domain comprising an amino acid sequence having 80, 81, 82, 83, 84, 85, 86, 87, 88, 89, 90, 91, 92, 93, 94, 95, 96, 97, 98, or 99% identity to SEQ ID NO: 8 and comprising LCDR1 comprising the amino acid sequence SEQ ID NO: 4, LCDR2 comprising the amino acid sequence SEQ ID NO: 5, and LCDR3 comprising the amino acid sequence SEQ ID NO: 6. In some embodiments, the anti-IL-25 antibody or antigen binding fragment thereof can also comprise at least one immunoglobulin light chain variable domain comprising an amino acid sequence having 80, 81, 82, 83, 84, 85, 86, 87, 88, 89, 90, 91, 92, 93, 94, 95, 96, 97, 98, or 99% identity to SEQ ID NO: 16 and comprising LCDR1 comprising the amino acid sequence SEQ ID NO: 12, LCDR2 comprising the amino acid sequence SEQ ID NO: 13, and LCDR3 comprising the amino acid sequence SEQ ID NO: 14.

In some embodiments, the anti-IL-25 antibody or antigen binding fragment thereof for use in the methods of treatment disclosed herein comprises a humanized version of LNR-125 or an antigen binding fragment thereof, as disclosed in the U.S. Pat. No. 11,492,397, hereby incorporated in its entirety by reference. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some embodiments, the anti-IL-25 antibody is fully humanized wherein all the framework residues are derived from human immunoglobulins (recipient antibody). In some embodiments, the anti-IL-25 antibody is partially humanized. In some instances, framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. Methods for humanizing non-human antibodies are well known in the art.

In some embodiments, the anti-IL-25 antibody or antigen binding fragment thereof for use in the methods of treatment disclosed herein comprises an antigen binding site comprising at least one immunoglobulin heavy chain variable domain comprising SEQ ID NO: 17 (QVQLVQSGAEVKKPGASVKVSCKASGYTFSSYWIEWVRQAPGQGLEWIGQILPGIG STNYNEKFKGRVTITADTSTSTVYMELSSLRSEDTAVYYCARGYGNYGDYWGQGTT VTVSS) at least one immunoglobulin light chain variable domain comprising SEQ ID NO: 18 (DIVLTQSPASLAVSPGQRATITCRASESVDSYGNSFMHWYQQKPGQPPKLLIYRASN LESGVPARFSGSGSGTDFTLTINPVEAQDTANYYCQQSNEDPLTFGAGTKLELKR). In some embodiments, the anti-IL-25 antibody or antigen binding fragment thereof comprises an antigen binding site comprising at least one immunoglobulin heavy chain variable domain comprising an amino acid sequence having 80, 81, 82, 83, 84, 85, 86, 87, 88, 89, 90, 91, 92, 93, 94, 95, 96, 97, 98, or 99% identity to SEQ ID NO: 17. In some embodiments, the anti-IL-25 antibody or antigen binding fragment thereof comprises an antigen binding site comprising at least one immunoglobulin light chain variable domain comprising an amino acid sequence having 80, 81, 82, 83, 84, 85, 86, 87, 88, 89, 90, 91, 92, 93, 94, 95, 96, 97, 98, or 99% identity to SEQ ID NO: 18.

In some embodiments, the anti-IL-25 antibody for use in the methods of treatment disclosed herein comprises 22C7 or an antigen binding fragment thereof disclosed in Bone R et al., Discovery and multi-parametric optimization of a high-affinity antibody against Interleukin-25 with neutralizing activity in a mouse model of skin inflammation, Antibody Therapeutics Vol. 5/Issue 4 pp. 258-267 (October 2022), hereby incorporated in its entirety by reference.

In some embodiments, the antigen binding fragment of an anti-IL-25 antibody comprises fragments, such as F(ab′)2, Fab′, Fab, Fv, sFv, dAb, complementarity determining region (CDR) fragments, single-chain antibodies (scFv), bivalent single-chain antibodies, diabodies, triabodies, tetrabodies, (poly)peptides that contain at least a fragment of an immunoglobulin that is sufficient to confer specific antigen binding to the (poly)peptide, etc., including hybrid fragments. Thus, fragments of the antibodies that retain the ability to bind IL-25 specific antigens are provided. Such antibodies and fragments can be made by techniques known in the art and can be screened for specificity and activity according to the methods known in the art.

In some embodiments, the nucleic acid sequence of the anti-IL-25 antibody codes for an amino acid sequence that comprises at least a variable heavy and variable light chain portions of the amino acid sequence of the anti-IL-25 antibodies described herein. In some embodiments, the nucleic acid sequence encoding the anti-IL-25 antibody codes for an amino acid sequence that comprises at least the CDRs of the variable heavy chain and the CDRs of the variable light chain portions of the amino acid sequence of the anti-IL-25 antibodies described herein.

In some embodiments, the anti-IL-25 antibody or antigen binding fragment thereof comprises conjugated antibodies or antibody fragments. Conjugated antibodies or fragments refer to antibodies or fragments that are operatively linked or otherwise physically or functionally associated with an effector moiety or tag, such as inter alia a toxic substance, a radioactive substance, fluorescent substance, a liposome, or an enzyme. In some embodiments, the anti-IL-25 antibody or antigen binding fragment thereof is conjugated to a nanoparticle which can comprise a payload. Exemplary payloads include, but are not limited to, dexamethasone and budesonide, IL-2, and IL-15. In some embodiments, the dexamethasone or budesonide treats irAEs. In some embodiments, IL-2 or IL-15 treats cancer.

### Antibody Production

The antibodies disclosed herein can be produced by any method known in the art. In some embodiments, the antibodies disclosed herein are produced by culturing a cell transfected or transformed with a vector comprising nucleic acid sequences encoding an antibody described herein and isolating the antibody.

In some embodiments, antibodies are synthesized by the hybridoma culture method which results in antibodies that are not contaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques known in the art, including, for example, the hybridoma method (e.g., Kohler and Milstein., Nature, 256:495-97 (1975); Hongo et al, Hybridoma, 14 (3): 253-260 (1995), Harlow et al, Antibodies: A Laboratory Manual, (Cold Spring Harbor Laboratory Press, 2nd ed. 1988); Hammerling et al, in: Monoclonal Antibodies and T-Cell Hybridomas 563-681 (Elsevier, N. Y., 1981)), recombinant DNA methods, phage-display technologies (see, e.g., Clackson et al, Nature, 352: 624-628 (1991); Marks et al, J. Mol Biol. 222: 581-597 (1992); Sidhu et al, J. Mol Biol. 338(2): 299-310 (2004); Lee et al, J. Mol Biol. 340(4): 1073-1093 (2004); Fellouse, Proc. Natl. Acad. ScL USA 101(6): 12467-12472 (2004); and Lee et al, J. Immunol. Methods 284(1-2): 119-132 (2004), and technologies for producing human or humanlike antibodies in animals that have parts or all of the human immunoglobulin loci or genes encoding human immunoglobulin sequences (see, e.g., Lonberg et al, Nature 368: 856-859 (1994); Morrison, Nature 368: 812-813 (1994); Fishwild et al, Nature Biotechnol 14: 845-851 (1996); Neuberger, Nature Biotechnol. 14: 826 (1996); and Lonberg and Huszar, Intern. Rev. Immunol. 13: 65-93 (1995).

In some embodiments, expression of an antibody comprises expression vector(s) containing a polynucleotide that encodes an antibody described herein. Methods that are well known to those skilled in the art can be used to construct expression vectors comprising antibody coding sequences and appropriate transcriptional and translational control signals. These methods include, for example, in vitro recombinant DNA techniques, synthetic techniques, and in vivo genetic recombination. Particular embodiments provide replicable vectors comprising a nucleotide sequence encoding an a antibody disclosed herein operably linked to a promoter. In preferred embodiments, such vectors may include a nucleotide sequence encoding the heavy chain of an antibody molecule (or fragment thereof), a nucleotide sequence encoding the light chain of an antibody (or fragment thereof), or both the heavy and light chain.

The polynucleotide encoding the antibody may be modified, for example, by substituting the coding sequence for human heavy- and light-chain constant domains in place of the homologous murine sequences (U.S. Pat. No. 4,816,567; Morrison, et al, Proc. Natl Acad. ScL USA, 81:6851 (1984)), or by covalently joining to the immunoglobulin coding sequence all or part of the coding sequence for a non-immunoglobulin polypeptide. Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen. The monoclonal antibodies described herein may by monovalent, the preparation of which is well known in the art. For example, one method involves recombinant expression of immunoglobulin light chain and a modified heavy chain. The heavy chain is truncated generally at any point in the Fc domain so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art. Chimeric or hybrid antibodies also may be prepared in vitro using known methods in synthetic protein chemistry, including those involving crosslinking agents.

Various expression systems for producing antibodies are known in the art, and include, prokaryotic (e.g., bacteria), plant, insect, yeast, and mammalian expression systems. Suitable cell lines, can be transformed, transduced, or transfected with nucleic acids containing coding sequences for antibodies or portions of antibodies disclosed herein in order to produce the antibody of interest. Expression vectors containing such nucleic acid sequences, which can be linked to at least one regulatory sequence in a manner that allows expression of the nucleotide sequence in a host cell, can be introduced via methods known in the art. Practitioners in the art understand that designing an expression vector can depend on factors, such as the choice of host cell to be transfected and/or the type and/or amount of desired protein to be expressed. Enhancer regions, which are those sequences found upstream or downstream of the promoter region in non-coding DNA regions, are also known in the art to be important in optimizing expression. If needed, origins of replication from viral sources can be employed, such as if a prokaryotic host is utilized for introduction of plasmid DNA. However, in eukaryotic organisms, chromosome integration is a common mechanism for DNA replication. For stable transfection of mammalian cells, a small fraction of cells can integrate introduced DNA into their genomes. The expression vector and transfection method utilized can be factors that contribute to a successful integration event. For stable amplification and expression of a desired protein, a vector containing DNA encoding a protein of interest (e.g., antibodies and fragments thereof) is stably integrated into the genome of eukaryotic cells (for example mammalian cells), resulting in the stable expression of transfected genes. A gene that encodes a selectable marker (for example, resistance to antibiotics or drugs) can be introduced into host cells along with the gene of interest in order to identify and select clones that stably express a gene encoding a protein of interest. Cells containing the gene of interest can be identified by drug selection wherein cells that have incorporated the selectable marker gene will survive in the presence of the drug. Cells that have not incorporated the gene for the selectable marker die. Surviving cells can then be screened for the production of the desired antibody molecule.

In some embodiments, the antibodies disclosed herein are encoded in a vector for expression in a cell line. In some embodiments, a vector comprises a polynucleotide sequence that encodes an anti-IL-25 antibody (or anti-PD-1, anti-PDL-1, or anti-CTLA-4 antibody) and the vector is transfected into one or more cell lines for expression. In some embodiments, one or more vectors comprise polynucleotide sequences encoding a light chain and a heavy chain of the antibody. For example, in some embodiments, a first vector may comprise a polynucleotide sequence encoding a light chain, a second vector may comprise a polynucleotide sequence encoding a heavy chain, of anti-IL-25 antibody (or anti-PD-1, anti-PDL-1, or anti-CTLA-4 antibody). In some embodiments, both vectors are transfected into one or more cell lines for expression. A host cell strain, which modulates the expression of the inserted sequences, or modifies and processes the nucleic acid in a specific fashion desired also may be chosen. Such modifications (for example, glycosylation and other post-translational modifications) and processing (for example, cleavage) of protein products may be important for the function of the antibody. Different host cell strains have characteristic and specific mechanisms for the post-translational processing and modification of proteins and gene products. As such, appropriate host systems or cell lines can be chosen to ensure the correct modification and processing of the foreign antibody expressed. Thus, eukaryotic host cells possessing the cellular machinery for proper processing of the primary transcript, glycosylation, and phosphorylation of the gene product may be used.

Various culturing parameters can be used with respect to the host cell being cultured. Appropriate culture conditions for mammalian cells are well known in the art (Cleveland W L, et al., J Immunol Methods, 1983, 56 (2): 221-234) or can be determined by the skilled artisan (see, for example, Animal Cell Culture: A Practical Approach 2nd Ed., Rickwood, D. and Hames, B. D., eds. (Oxford University Press: New York, 1992)). Cell culturing conditions can vary according to the type of host cell selected. Commercially available media can be utilized.

Antibodies disclosed herein can be purified from any human or non-human cell which expresses the antibody, including those which have been transfected with expression constructs that express the antibody or fragments thereof. For antibody recovery, isolation and/or purification, the cell culture medium or cell lysate is centrifuged to remove particulate cells and cell debris. The desired antibody molecule is isolated or purified away from contaminating soluble proteins and polypeptides by suitable purification techniques. Non-limiting purification methods for proteins/antibodies include: size exclusion chromatography; affinity chromatography; ion exchange chromatography; ethanol precipitation; reverse phase HPLC; chromatography on a resin, such as silica, or cation exchange resin, e.g., DEAE; chromatofocusing; SDS-PAGE; ammonium sulfate precipitation; gel filtration using, e.g., Sephadex G-75, Sepharose; protein A sepharose chromatography for removal of immunoglobulin contaminants; and the like. Other additives, such as protease inhibitors (e.g., PMSF or proteinase K) can be used to inhibit proteolytic degradation during purification. Purification procedures that can select for carbohydrates can also be used, e.g., ion-exchange soft gel chromatography, or HPLC using cation- or anion-exchange resins, in which the more acidic fraction(s) is/are collected.

### IL-25 Antagonists

In certain aspects, described herein is a method for treating cancer in a subject in need thereof comprising administering to the subject a composition comprising a therapeutically effective amount of an anti-IL-25 antagonist.

In some embodiments, the composition comprises an IL-25 small interfering ribonucleic acid (siIL25). In some embodiments, the siIL25 comprises the sequences encoding the small interfering ribonucleic acid (siIL-25) of any of the sequences of Table 1, of SEQ ID NO:9 (ctagtgtagttactagtcttttgaca), or of SEQ ID NO:10 (atttgtttgtttactcatcactcag). In various embodiments, the siRNA comprises a nucleic acid sequence at least 75%, 80%, 85%, 90%, 95%, 96%, 97%, 98% or 99% identical to any of the sequences of Table 1, of SEQ ID NO:9 (ctagtgtagttactagtcttttgaca), or of SEQ ID NO:10 (atttgtttgtttactcatcactcag). In some embodiments, the siRNA consists of a siRNA nucleic acid sequence of any of the sequences of Table 1.

TABLE 1

| SEQ. ID No. | siIL-25 |
|---|---|
| 19 | tgtaccgtgtttccttagcttgtgt |
| 20 | ggcgagaagggcacccacaagggct |
| 21 | agcagctgctgcttagggccgccgg |
| 22 | gggcacccacaagggctactgcctg |
| 23 | tccccctggagatatgagttggaca |
| 24 | gtgtacaaccacttgccatgaaggg |
| 25 | cgcaggctgtaccgtgtttccttag |
| 26 | gcccatttctggaggccaccactcc |
| 27 | gggctactgcctggagcgcaggctg |
| 28 | ccaggtgtacaaccacttgccatga |
| 29 | ccggggcaactcggagctgctctac |
| 30 | ctgtaccgtgtttccttagcttgtg |
| 31 | atgccatggcgagaagggcacccac |
| 32 | atgccatggcgagaagggcacccac |
| 33 | cagctgctgcttagggccgccggaa |
| 34 | gcacccacaagggctactgcctgga |
| 35 | cccctggagatatgagttggacaga |
| 36 | gtacaaccacttgccatgaagggcc |
| 37 | gctgtaccgtgtttccttagcttgt |
| 38 | aaagttctgcccatttctggaggcc |
| 39 | ggctactgcctggagcgcaggctgt |
| 40 | caggtgtacaaccacttgccatgaa |
| 41 | ccggggcaactcggagctgctctac |

In some embodiments, the composition comprises an IL-25 short-hairpin ribonucleic acid (shIL-25). In some embodiments, the shIL-25 comprises a nucleic acid sequences of any of the sequences of Table 1, of SEQ ID NO:9 (ctagtgtagttactagtcttttgaca), or of SEQ ID NO:10 (atttgtttgtttactcatcactcag). In some embodiments, the composition comprises a viral vector comprising a nucleic acid sequence encoding a shIL-25. In some embodiments, the viral vector is an adeno-associated vector (AAV). In various embodiments, the viral vector is a vector that preferentially targets the liver or liver cells. In various embodiments, the AAV is AAV 1, 2, 3, 4, 5, 6, 7, 8, 9, or 10 or variant thereof. In some embodiments, the viral vector is AAV8.

Inhibition of RNA encoding IL-25 can effectively modulate the expression of these proteins. Inhibitors can include shRNAs encoding siRNAs, siRNA; interfering RNA or RNAi; dsRNA; RNA Polymerase III transcribed DNAs; ribozymes; Oligonucleotide (ASO) and antisense nucleic acids, which can be RNA, DNA, or an artificial nucleic acid.

Antisense oligonucleotides, including antisense DNA, RNA, and DNA/RNA molecules, act to directly block the translation of mRNA by binding to targeted mRNA and preventing protein translation. For example, antisense oligonucleotides of at least about 15 bases and complementary to unique regions of the DNA sequence encoding an EGFR fusion molecule can be synthesized, e.g., by conventional phosphodiester techniques. Antisense nucleotide sequences include, but are not limited to: morpholinos, 2′-O-methyl polynucleotides, DNA, RNA and the like.

siRNA comprises a double stranded structure containing from about 15 to about 50 base pairs, for example from about 21 to about 25 base pairs, and having a nucleotide sequence identical or nearly identical to an expressed target gene or RNA within the cell. The siRNA comprise a sense RNA strand and a complementary antisense RNA strand annealed together by standard Watson-Crick base-pairing interactions. The sense strand comprises a nucleic acid sequence which is substantially identical to a nucleic acid sequence contained within the target miRNA molecule. “Substantially identical” to a target sequence contained within the target mRNA refers to a nucleic acid sequence that differs from the target sequence by about 3% or less. The sense and antisense strands of the siRNA can comprise two complementary, single-stranded RNA molecules, or can comprise a single molecule in which two complementary portions are base-paired and are covalently linked by a single-stranded “hairpin” area.

The siRNA can be altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribo-nucleotides. One or both strands of the siRNA can also comprise a 3′ overhang. As used herein, a 3′ overhang refers to at least one unpaired nucleotide extending from the 3′-end of a duplexed RNA strand. For example, the siRNA can comprise at least one 3′ overhang of from 1 to about 6 nucleotides (which includes ribonucleotides or deoxyribonucleotides) in length, or from 1 to about 5 nucleotides in length, or from 1 to about 4 nucleotides in length, or from about 2 to about 4 nucleotides in length. For example, each strand of the siRNA can comprise 3′ overhangs of dithymidylic acid (“TT”) or diuridylic acid (“uu”).

siRNA can be produced chemically or biologically, or can be expressed from a recombinant plasmid or viral vector. Methods for producing and testing dsRNA or siRNA molecules are known in the art. A short hairpin RNA (shRNA) encodes an RNA molecule with a tight hairpin turn that can be used to silence target gene expression via RNA interference (RNAi). Expression of shRNA in cells is typically accomplished by delivery of plasmids or through viral or bacterial vectors.

RNA polymerase III transcribed DNAs contain promoters, such as the U6 promoter. These DNAs can be transcribed to produce small hairpin RNAs in the cell that can function as siRNA or linear RNAs, which can function as antisense RNA. The IL-25 inhibitor can comprise ribonucleotides, deoxyribonucleotides, synthetic nucleotides, or any suitable combination such that the target RNA and/or gene is inhibited. In addition, these forms of nucleic acid can be single, double, triple, or quadruple stranded.

### Dosage

A prophylactically effective or therapeutically effective amount is typically dependent on the weight of the subject being treated, the subject's physical condition, the extensiveness of the condition to be treated, and the age of the subject being treated. In general, an anti-IL-25 antibody, or polynucleotides encoding one or more antibodies, disclosed herein may be administered in a therapeutically effective amount. In general, an anti-IL-25 antibody, or polynucleotides encoding one or more antibodies, disclosed herein may be administered in an amount in the range of about 10 ng/kg body weight to about 100 mg/kg body weight per dose. In some embodiments, antibodies may be administered in an amount in the range of about 50 μg/kg body weight to about 5 mg/kg body weight per dose. In some embodiments, antibodies may be administered in an amount in the range of about 100 μg/kg body weight to about 10 mg/kg body weight per dose. In some embodiments, antibodies may be administered in an amount in the range of about 100 μg/kg body weight to about 20 mg/kg body weight per dose. In some embodiments, antibodies may be administered in an amount in the range of about 0.5 mg/kg body weight to about 20 mg/kg body weight per dose. In some embodiments, antibodies may be administered in an amount in the range of about 0.5 mg/kg body weight to about 10 mg/kg body weight per dose. In some embodiments, antibodies may be administered in an amount in the range of about 1 mg/kg body weight to about 5 mg/kg body weight per dose. In some embodiments, antibodies may be administered in an amount in the range of about 0.1 mg/kg body weight to about 0.5 mg/kg body weight per dose. In some embodiments, antibodies may be administered in a dose of at least about 100 μg/kg body weight, at least about 250 μg/kg body weight, at least about 500 μg/kg body weight, at least about 750 μg/kg body weight, at least about 3 mg/kg body weight, at least about 5 mg/kg body weight, or at least about 10 mg/kg body weight.

In some methods, the dosage is adjusted to achieve a plasma antibody concentration of about 1-1000 μg/mL or about 25-300 μg/mL. In some embodiments, the dosage is adjusted to achieve a plasma antibody concentration of about 0.001 μg/mL to about 10 μg/mL. In some embodiments, the dosage is adjusted to achieve a plasma antibody concentration of about 1 μg/mL to about 10 μg/mL. In some embodiments, the dosage is adjusted to achieve a plasma antibody concentration of about 0.01 μg/mL to about 1 μg/mL. In some embodiments, the dosage is adjusted to achieve a plasma antibody concentration of about 0.01 μg/mL to about 0.1 μg/mL.

In general, an anti-IL-25 antibody, or polynucleotides encoding one or more antibodies, disclosed herein may be administered in a therapeutically effective amount.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1: Anti-IL-25 Monoclonal Antibody Inhibits Tumor Growth in a Breast Cancer Model

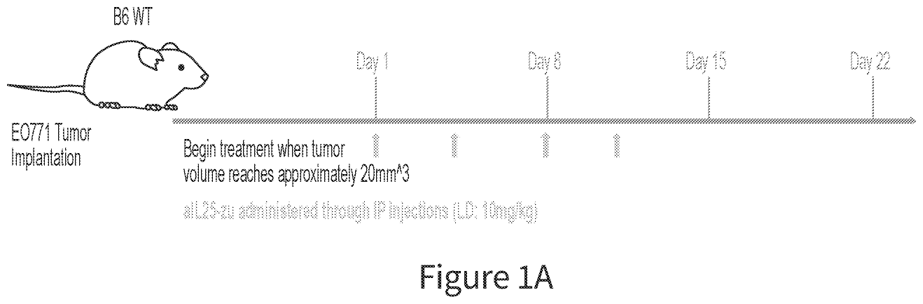

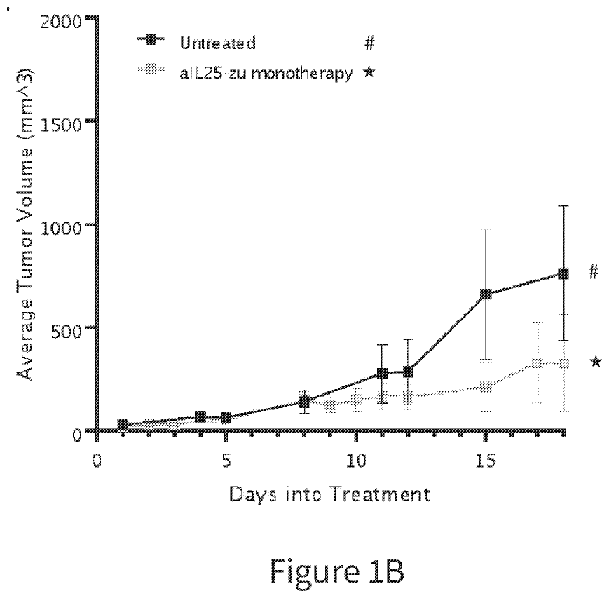

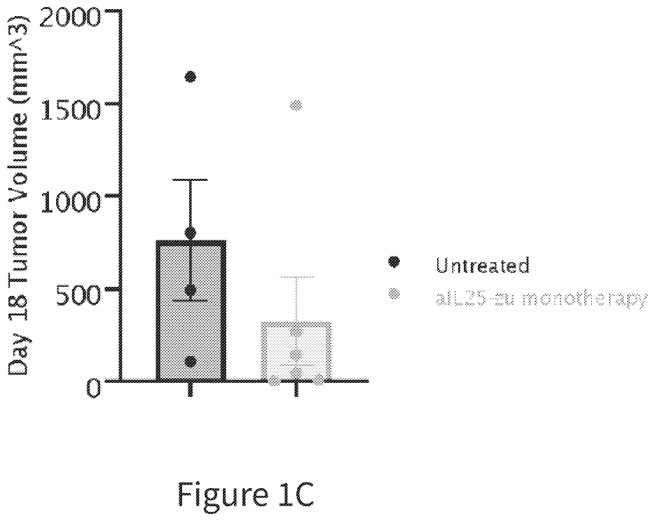

The effect of neutralizing IL-25 in treating cancer was examined. In the EO771 experimental model, B6 mice inoculated with EO771 (medullary breast adenocarcinoma) tumor cells received an anti-IL-25-zu antibody mono-treatment experiment. Anti-IL25-zu is anti-IL25 with humanized Fc and is also referred to as LNR125.38. Tumor growth inhibition in the EO771 syngeneic tumor model was also demonstrated. Remarkably, treatment with clone aIL25-zu alone inhibited tumor growth (FIGS. 1A-C). Quantification of average tumor volume showed a significant decrease in tumor volume over time (FIGS. 1B-C).

Described herein is the administration of anti-IL25 antibodies as a monotherapy to reduce tumor size. These findings suggest that IL25 can serve a role in treating tumors.

### Example 2: Anti-IL-25 Monoclonal Antibody Inhibits Tumor Growth in Tumor Models

Mice (e.g., B6/lpr female mice (Jax)) are inoculated with tumor cells of the following cell lines: LL/2, 4T1, Hepa 1-6, PTEN-CaP8, Hs 835.T, LMH, CT26, MtC-CaP, MEL11443, B2905, EMT6, PLHC-1, KLN 205, MBT-2, MC38, GL261, C1498, KLN205, B16b Clone M-3, NIE, 4T1, EMT6, JC, Renca, H22, A20, EL4, MPC-11, Pan02, RM-1, C1498, H22. Tumor cells can be injected into the flank. Tumor volumes are measured daily with mechanical caliper. Mice are treated with anti-IL-25 (e.g., LNR125.38) alone or untreated. Anti-human IL-25 (e.g., LNR125.38) is given intra peritoneally at 10 mg/kg starting on day 1, when tumor volumes were between 20 to 30 mm\hat{ }3, twice a week for two weeks (4 treatments in total). Administration of anti-IL-25 alone can suppress tumor growth and increase survival of animals.

METHODS

| Material | Source | Catalog Number |
|---|---|---|
| Mice | | |
| B6 Lpr mouse | The Jackson Laboratory | 000482 |
| B6 mouse | The Jackson Laboratory | 000664 |
| In-Vivo Antibodies/Drugs | | |
| Murine/human cross reactive | Lanier Biotherapeutics | LNR125 and LNR125.38 |
| IL-25 antibody | | |
| Flow/IHC Antibodies | | |
| Alexa Fluor 488 anti-mouse | BioLegend | 100210 |
| CD3 | | |
| Brilliant Violet 510 anti- | BioLegend | 100553 |
| mouse CD4 | | |
| PerCP/Cy5.5 anti-mouse | BioLegend | 100734 |
| CD8a | | |
| Brilliant Violet 421 anti- | BioLegend | 103040 |
| mouse/human CD44 | | |
| Brilliant Violet 711 anti- | BioLegend | 104445 |
| mouse CD62L | | |
| APC anti-mouse CD69 | BioLegend | 104514 |
| CD3 antibody [SP7] | GeneTex | GTX16669 |
| Reagents | | |
| DMEM, 1X | Corning | 10-013-CVvc |
| HI FBS | Gibco | 10438-026 |
| Penicillin Streptomycin | Corning | 30-001-CI |
| HEPES Buffer | Corning | 25-060-CI |
| PBS, 1X | Corning | 21-040-CV |
| 0.25% Trypsin | Corning | 25-053-CI |
| Dimethyl Sulfoxide | Fisher Bioreagents | BP231-1 |
| Lymphoprep | STEM CELL Technologies | 07801 |
| RPMI 1640, 1X | Corning | 10-040-CVR |
| ACK Lysing Buffer | Gibco | A10492-01 |
| TruStain Monocyte Blocker | BioLegend | 426103 |
| TruStain FcX ™ PLUS (anti- | BioLegend | 156604 |
| mouse CD16/32) Antibody | | |
| TruStain FcX ™ (anti-mouse | BioLegend | 101320 |
| CD16/32) Antibody | | |
| Brilliant Stain Buffer | BD Horizon | 563794 |
| 10% Neutral Buffered | Thermo Scientific | 5701 |
| Formalin | | |
| Ethanol, Anhydrous | Fisher Bioreagents | A405P-4 |
| Kits | | |
| Mycoplasma Detection Kit | InvivoGen | rep-mys-50 |
| LIVE/DEAD ™ Fixable Blue | Invitrogen | L34962 |
| Dead Cell Stain Kit, for UV | | |
| excitation | | |
| True-Nuclear ™ | BioLegend | 424401 |
| Transcription Factor Buffer | | |
| Set | | |
| Zombie UV ™ Fixable | BioLegend | 423107 |
| Viability Kit | | |
| IL-25 (IL-17E) Mouse | Invitrogen | EPX01A-26046-901 |
| ProcartaPlexTM Simplex Kit | | |
| Cytokine & Chemokine 36- | Invitrogen | EPX360-26092-901 |
| Plex Mouse 1 | | |
| ProcartaPlexTM Panel 1A | | |
| Software/Computer Based | | |
| Tools | | |
| HALO | Indica Labs | |
| FlowJo_V10 | BD Biosciences | |
| UMAP | Scikit-Learn | |
| Python | Python Software Foundation | |
| Kaplan-Meier Plotter | KMPlot.com | |
| Prism 9 | GraphPad | |

### Cell Culture

The murine breast cancer cell line EO771 (Robert F. Schwabe) was cultured in DMEM medium (Corning) with 10% FBS (Gibco), 1% Pen-Step (Corning), and 20 mM HEPES (Corning). Cells were grown in a 37° C. incubator and routinely examined for mycoplasma using mycoplasma detection kit (InvivoGen).

### Mice Breeding

B6 Lpr mice purchased from JAX (Cat #) were housed in the Columbia Institute of Comparative Medicine animal facility under protocol AC-AABO7553. Breeding cages were set up with two females and one male. Litters are routinely genotyped through PCR using Fas gene primers—oIMR1678 (5′-GTA AAT AAT TGT GCT TCG TCA G-3′) as common primer, oIMR1679 (5′-TAG AAA GGT GCA CGG GTG TG-3′) for Fas^{Lpr} mutant, and oIMR1680 (5′-CAA ATC TAG GCA TTA ACA GTG-3′) for Fas^{WT}. Mutant mice with homozygote alleles were recruited as new breeders or used for experiments. B6 WT mice were purchased from JAX and used directly.

Intraperitoneal anti-IL25-zu (Lanier) were administered. The order of mice being treated and measured was random.

For the breast cancer tumor study, 2×10⁵ EO771 cells were suspended in cold, sterile PBS (Corning) and implanted subcutaneously into the right flank of 8-week-old wild-type B6 mice. Tumor volumes were tracked. Once the tumor reached 20-50 mm³, intraperitoneal anti-IL25-zu (200 μg biweekly) treatment was initiated in the experimental group (n=6) and continued for two weeks. Control group mice (n=4) received no treatment.

The cages were housed on the same rack to minimize potential confounder variables. Tumors and animal health have been closely monitored by researchers (not blinded) and veterinarians at Columbia Institute of Comparative Medicine. We provided humane endpoint euthanasia when tumor volume reached over 2000 mm³ or when tumor became extremely ulcerated.

### Luminex Assay and Analysis

Peripheral blood was collected from the heart of the mice post-euthanizing. Blood serum was isolated by centrifugation at 10,000×g for 10 minutes. Isolated serum was stored at −80° C. before the Luminex assay. Columbia Biomarkers Core Laboratory performed Luminex magnetic bead assay using 36-plex mouse panel (Invitrogen) and IL-25 simplex (Invitrogen) kits. Each sample was run in duplicates. The coefficient of variation (CV) between duplicated samples was calculated. Repeated samples with CV>20% were eliminated. Models with cytokine/chemokine levels below the lower level of detection (LLOD) were assigned a value equal to LLOD/√2. Samples with cytokine/chemokine levels above the upper level of detection (ULOD) were given the value of ULOD. Cytokines/chemokines with more than 40% samples of undetectable values were discarded. Cytokine/chemokine values underwent a natural log transformation for normal distributions. Samples outside of their treatment groups' respective 95% CI were eliminated. The data presented only shows stratified data of interest.

### Histology Study

At the in-vivo endpoint, mice were transcardially perfused with 10 ml saline to clear blood. Heart, liver, lung, colon, pancreas, and tumor were collected and washed in 10 ml PBS before being transferred to 10 ml 10% formalin (Thermo Scientific). After >24 hours of fixation in formalin, the tissues were transferred to 70% histology-grade anhydrous ethanol (Fisher Bioreagents). Samples were then sent to Columbia Molecular Pathology Shared Resource (MPSR) for slicing, hematoxylin-eosin (H&E) stain, and paraffin embedding. H&E slides were viewed under a light microscope. Immune cell infiltration severity scores were graded by two trained experts on a scale of 0-3 (5).

### Immunohistochemistry

Immune-blank slides are made from paraffin-embedded blocks and stained with anti-CD3 (GeneTex) by HistoWiz. Scanned images of IHC slides are processed using HALO (Indica Labs) for artificial intelligence CD3⁺ T-cell labeling (7).

### Flow Cytometry and Gating Strategy

Livers and tumors were harvested at the endpoint of in-vivo experiments after perfusion. Livers were smashed through 100 μM filters using syringe plungers and collected in 20 ml of FACS buffer (2% FBS in PBS) in a 50 ml centrifuge tube. Liver cells pellet after centrifugation at 50×g for 2 minutes (brake off). The supernatant was collected and washed with PBS. Tumors were diced into small pieces using a scalpel blade and transferred to a 5 ml digestion mixture (500 ml PBS+500 mg collagenase D+25 ml FCS+10 mg DNase) in 15 ml conical tubes. The samples were incubated in a 37° C. water bath for 30 minutes. Digested tissue samples were vortexed vigorously before being smashed through 45 μM filters and collected using 5 ml RPMI medium (Corning) containing 10% FBS and 1% Pen-Strep. Following centrifugation and collection, the cells were washed once with PBS. Liver and tumor lymphocytes were isolated using Lymphoprep (Stem Cell Technologies) following Lymphoprep's standard protocol. Isolated lymphocytes were washed with PBS and used immediately for flow cytometry. Lymphocytes isolated from the liver and tumor were stained for 34-plex flow cytometry using a protocol consisting of four parts: (I) live/dead staining, (II) surface staining, (III) fixation/permeabilization, and (iv) intracellular staining. Live/dead stain was performed with a live/dead fixable blue dead cell stain kit (Invitrogen). Following live/dead colors, Fc receptors, and monocytes were blocked using TruStain FcX PLUS (BioLegend) and TruStain Monocyte Blocker (BioLegend). Then, the cells were stained for cell surface proteins using fluorophore conjugated antibodies: TCR-B BUV395, CD103 BUV496, CD44 BUV563, PD-1 BUV615, Nrp1 BUV661, CD4 BUV805, CD39 BV421, IA-IE PacBlue, ST2 BV480, CD8 PacOrange, CD62L BV570, CD11c BV605, ICOS BV650, CXCR3 BV711, KLRG1 BV750, PD-L1 BV785, CD45 A532, Sca-1 PerCP, Ly6C PerCP-Cy5.5, CD206 PerCPeF710, NK1.1 PE-Cy5, B220 PE/Fire 810, CD69 SN685, CD11b A700, F4/80 APC-Fire750, and CD38 APC-Fire810. Following cell surface staining, the cells were fixed/permeabilized using a Fixation/Permeabilization Buffer Set (BioLegend). Post-fix and perm, the cells were stained for intracellular proteins using fluorophore-conjugated antibodies: Ki67 BUV737, iNOS FITC, TOX PE, Ly6G SYG593, Helios PE-Dazzle594, FoxP3 PE-Cy7, and TCF-1 APC. Data were acquired using Cytek 5L Aurora and analyzed using FlowJo and Python UMAP. Spleen was harvested at the endpoint of in-vivo experiments. Spleen tissue was smashed through 70 μM filters, washed twice in FACS, resuspended in ACK lysis buffer, and washed twice in FACS. Spleen-mixed white blood cells were used immediately for flow cytometry or frozen with 20% DMSO (Fisher Bioreagents) in FBS (Gibco). Dead cells in the mixed white blood cells isolated from the spleens were stained using Zombie UV™ Fixable Viability Kit, while the Fc receptors were blocked using TruStain FcX (BioLegend). Cell surface proteins were stained with C fluorophore-conjugated-antibodies, CD3 AF488 (BioLegend), CD4 BV510 (BioLegend), CD8 Percp/Cy5.5 (BioLegend), CD44 BV421 (BioLegend), CD62L BV711 (BioLegend), PD-1 PE-Cy7 (BioLegend), and CD69 APC, for CD4+/CD8+ T-cell identification, T_{Naive}/T_{CM}/T_{EM}/TEMRA subset differentiation, and activation/exhaustion observation. Data were recorded using Cytek 5L Aurora and analyzed with FlowJo.

### Kaplan-Meier Plots

### Statistical Analysis

Data were analyzed using Prism 9 (GraphPad) and presented as mean or mean±SEM. For comparison analysis of immune infiltration/cytokine level/tumor volume on day 18 bar graphs, an unpaired two-tailed t-test was performed. Additionally, an unpaired t-test performed on tumor volume on day 18 used Welch's correction. Survival probabilities were analyzed using the Kaplan-Meier estimate.

### REFERENCES

1 Powles, T., J. P. Eder, G. D. Fine, F. S. Braiteh, Y. Loriot, C. Cruz, J. Bellmunt, H. A. Burris, D. P. Petrylak, S. Teng, X. Shen, Z. Boyd, P. S. Hegde, D. S. Chen, and N. J. Vogelzang. 2014. MPDL3280A (anti-PD-L1) treatment leads to clinical activity in metastatic bladder cancer. Nature 515: 558-562.
2. Duan, J., L. Cui, X. Zhao, H. Bai, S. Cai, G. Wang, Z. Zhao, J. Zhao, S. Chen, J. Song, C. Qi, Q. Wang, M. Huang, Y. Zhang, D. Huang, Y. Bai, F. Sun, J. J. Lee, Z. Wang, and J. Wang. 2020. Use of Immunotherapy With Programmed Cell Death 1 vs Programmed Cell Death Ligand 1 Inhibitors in Patients With Cancer: A Systematic Review and Meta-analysis. JAMA Oncol. 6: 375-384.
3. Kuol, N., L. Stojanovska, K. Nurgali, and V. Apostolopoulos. 2017. The mechanisms tumor cells utilize to evade the host's immune system. Maturitas 105: 8-15.
4. Kuol, N., L. Stojanovska, K. Nurgali, and V. Apostolopoulos. 2017. The mechanisms tumor cells utilize to evade the host's immune system. Maturitas 105: 8-15.
5. Adam, K., A. Iuga, A. S. Tocheva, and A. Mor. 2021. A novel mouse model for checkpoint inhibitor-induced adverse events. PloS One 16: e0246168.
6. Zhou, Y., Y. B. Medik, B. Patel, D. B. Zamler, S. Chen, T. Chapman, S. Schneider, E. M. Park, R. L. Babcock, T. T. Chrisikos, L. M. Kahn, A. M. Dyevoich, J. E. Pineda, M. C. Wong, A. K. Mishra, S. H. Cass, A. P. Cogdill, D. H. Johnson, S. B. Johnson, K. Wani, D. A. Ledesma, C. W. Hudgens, J. Wang, M. A. Wadud Khan, C. B. Peterson, A. Y. Joon, W. Peng, H. S. Li, R. Arora, X. Tang, M. G. Raso, X. Zhang, W. C. Foo, M. T. Tetzlaff, G. E. Diehl, K. Clise-Dwyer, E. M. Whitley, M. M. Gubin, J. P. Allison, P. Hwu, N. J. Ajami, A. Diab, J. A. Wargo, and S. S. Watowich. 2023. Intestinal toxicity to CTLA-4 blockade driven by IL-6 and myeloid infiltration. J. Exp. Med. 220: e20221333.
7. Adam, K., and A. Mor. 2022. Immunohistochemistry of Immune Cells and Cells Bound to in vivo Administered Antibodies in Liver, Lung, Pancreas, and Colon of B6/lpr Mice. Bio-Protoc. 12: e4468.

What is claimed is:

1. A method for treating cancer in a subject in need thereof comprising administering to the subject a therapeutically effective amount of an anti-IL-25 antibody or antigen binding fragment thereof.

2. The method of claim 1, wherein the anti-IL-25 antibody or antigen binding fragment thereof is administered as a monotherapy.

3. The method of claims 1-2, wherein the anti-IL-25 antibody or antigen binding fragment thereof is a monoclonal antibody or antigen binding fragment thereof.

4. The method of claims 1-2, wherein the anti-IL-25 antibody or antigen binding fragment thereof is LNR-125 or antigen binding fragment thereof.

5. The method of claims 1-2, wherein the anti-IL-25 antibody or antigen binding fragment thereof is LNR-125.38 or antigen binding fragment thereof.

6. The method of claims 1-2, wherein the anti-IL-25 antibody or antigen binding fragment thereof is a humanized form of LNR-125 or antigen binding fragment thereof.

7. The method of claims 1-2, wherein the anti-IL-25 antibody or antigen binding fragment thereof, comprises:
a first arm comprising a first variable heavy chain domain and a first variable light chain domain, wherein a portion of the first arm is capable of binding to a portion of an IL-25; and
a second arm comprising a second variable heavy chain domain and a second variable light chain domain, wherein a portion of the second arm is capable of binding to a portion of the IL-25 protein;
wherein the first and second arms each further comprise a fragment, crystallizable (Fc) domain.

8. The method of claim 7, wherein the first and second arms each further comprise a CH1 domain, a hinge domain, and a CL domain.

9. The method of claims 7-8, where the portion of IL-25 bound by the first arm and second arm is the same.

10. The method of claims 7-8, wherein:
the first variable heavy chain domain of the first arm is encoded by a first polypeptide chain;
the first variable light chain domain of the first arm is encoded by a second polypeptide chain;
the second variable heavy chain domain of the second arm is encoded by a third polypeptide chain;
the second variable light chain domain of the second arm is encoded by a fourth polypeptide chain; and
the first variable heavy chain domain and first variable light chain domain form a first IL-25 binding site and wherein the second variable heavy chain domain and second variable light chain domain form a second IL-25 binding site.

11. The method of claim 10, wherein the first and second IL-25 binding sites are the same.

12. The method of claim 10, wherein the first and third polypeptide chain each further encode a hinge domain, a CH1 domain, and the Fc domain, and wherein the second and fourth polypeptide chain each further encode a CL domain.

13. The method of claims 10-12, wherein the first and third polypeptide chains comprise the same sequence and the second and fourth polypeptide chains comprise the same sequence.

14. The method of claims 7-13, wherein the first and second variable heavy chain domain each comprises HCDR1 comprising SEQ ID NO: 1, HCDR2 comprising SEQ ID NO: 2, and HCDR3 comprising SEQ ID NO: 3 and wherein the first and second variable light chain domain each comprises LCDR1 comprising SEQ ID NO: 4, LCDR2 comprising SEQ ID NO: 5, and LCDR3 comprising SEQ ID NO: 6.

15. The method of claims 7-13, wherein the first and second variable heavy chain domain each comprises HCDR1 comprising SEQ ID NO: 9, HCDR2 comprising SEQ ID NO: 10, and HCDR3 comprising SEQ ID NO: 11 and the first and second variable light chain domain each comprises LCDR1 comprising SEQ ID NO: 12, LCDR2 comprising SEQ ID NO: 13, and LCDR3 comprising SEQ ID NO: 14.

16. The method of claim 14, wherein the first and second variable heavy chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 7 and wherein the first and second variable light chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 8.

17. The method of claim 14, wherein the first and second variable heavy chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 17 and wherein the first and second variable light chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 18.

18. The method of claim 15, wherein the first and second variable heavy chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 15 and wherein the first and second variable light chain domain each further comprise an amino acid sequence 80, 81, 82, 83, 84, 85, 86, 87, 88, 89, 90, 91, 92, 93, 94, 95, 96, 97, 98, or 99% identical to SEQ ID NO: 16.

19. The method of claims 7-13, wherein the first and second variable heavy chain domain each comprises an amino acid sequence of SEQ ID NO: 7 and wherein the first and second variable light chain domain each comprises an amino acid sequence of SEQ ID NO: 8, or wherein the first and second variable heavy chain domain each comprises an amino acid sequence of SEQ ID NO: 15 and the first and second variable light chain domain each comprises an amino acid sequence of SEQ ID NO: 16.

20. The method of claims 7-13, wherein the first and third polypeptide chain each comprises an amino acid sequence comprising SEQ ID NO: 7 and the second and fourth polypeptide chain each comprises an amino acid sequence comprising SEQ ID NO: 8 or wherein the first and third polypeptide chain each comprises an amino acid sequence comprising SEQ ID NO: 15 and the second and fourth polypeptide chain each comprises an amino acid sequence comprising SEQ ID NO: 16.

21. The method of claims 10-13 and 19-20, wherein the first and second polypeptide chains are linked by one or more covalent disulfide bonds and the third and fourth polypeptide chains are linked by one or more covalent disulfide bonds.

22. The method of claims 10-13 and 20-21, wherein the first and third polypeptide chains are linked by one or more covalent disulfide bonds.

23. The method of claims 7-13, wherein the first and second variable heavy chain domain each comprises an amino acid sequence of SEQ ID NO: 17, wherein the first and second variable light chain domain each comprises an amino acid sequence of SEQ ID NO: 18.

24. The method of claims 10-13, wherein the first and third polypeptide chain each comprises an amino acid sequence comprising SEQ ID NO: 17 and the second and fourth polypeptide chain each comprises an amino acid sequence comprising SEQ ID NO: 18.

25. The method of claim 24, wherein the first and second polypeptide chains are linked by one or more covalent disulfide bonds and the third and fourth polypeptide chains are linked by one or more covalent disulfide bonds.

26. The method of claims 24-25, wherein the first and third polypeptide chains are linked by one or more covalent disulfide bonds.

27. The method of claims 1-26, wherein the subject has a solid tumor.

28. The method of claims 1-27, wherein the cancer is lung carcinoma, breast cancer, hepatoma, Carcinoma of prostate gland, renal cell carcinoma, hepatocellular carcinoma, prostate cancer, melanoma, poeciliopsis lucida hepatocellular carcinoma, squamous cell carcinoma, bladder transitional cell carcinoma, glioma, myeloid leukemia, neuroblastoma tumor, stage IV breast cancer, mammary carcinoma cell line, renal cortical adenocarcinoma, B cell lymphoma, lymphoma, plasmacytoma, pancreatic cancer, or acute myeloid leukemia.

29. The method of claim 28, wherein the cancer is breast cancer.

30. The method of claim 28, wherein the cancer is melanoma.

31. The method of claim 28, wherein the cancer is ovarian cancer.

32. The method of claims 1-27, wherein the subject has a hematopoietic malignancy.

33. The method of claims 1-32, wherein the tumor of the subject is reduced in volume.

34. The method of claims 1-32, wherein growth of a tumor or cancer cells of the subject is inhibited.

35. The method of claims 1-34, wherein the subject is a human.


SEQUENCE LISTING (46 sequences)

SEQ ID NO: 1 - protein, 7 aa, synthetic construct
TSGMGVG

SEQ ID NO: 2 - protein, 16 aa, synthetic construct
HIWWDDVKRYNPALKS

SEQ ID NO: 3 - protein, 8 aa, synthetic construct
TLPHFFDY

SEQ ID NO: 4 - protein, 10 aa, synthetic construct
SASSSVSYMY

SEQ ID NO: 5 - protein, 7 aa, synthetic construct
RTSNLAS

SEQ ID NO: 6 - protein, 11 aa, synthetic construct
KQYHSYPPTWT

SEQ ID NO: 7 - protein, 118 aa, synthetic construct
QVTLKVSGPGILQPSQTLSLTCSFSGFSLNTSGMGVGWIRQPSGKGLEWLAHIWWDDVKRYNPALKSRLTISKDTSGSQVFLKIASVDTADTATYYCARTLPHFFDYWGQGTTLTVSS

SEQ ID NO: 8 - protein, 109 aa, synthetic construct
DIQMTQSPAIMSASPGEKVTISCSASSSVSYMYWYQQKSGSSPKPWIYRTSNLASGVPARFSGSGSGTSYSLTISSMEAEDAATYYCKQYHSYPPTWTFGGGTKLEIKR

SEQ ID NO: 9 - protein, 5 aa, synthetic construct
SYWIE

SEQ ID NO: 10 - protein, 17 aa, synthetic construct
QILPGIGSTNYNEKFKG

SEQ ID NO: 11 - protein, 8 aa, synthetic construct
GYGNYGDY

SEQ ID NO: 12 - protein, 14 aa, synthetic construct
RASESVDSYGNSFM

SEQ ID NO: 13 - protein, 7 aa, synthetic construct
RASNLES

SEQ ID NO: 14 - protein, 9 aa, synthetic construct
QQSNEDPLT

SEQ ID NO: 15 - protein, 117 aa, synthetic construct
EVKVVESGADLMKPGASVKISCKATGYTFSSYWIEWVKQRPGHGLEWIGQILPGIGSTNYNEKFKGKATFTADTSSNTAYMQLSSLTSEDSAVYYCARGYGNYGDYWGQGTTVTVSS

SEQ ID NO: 16 - protein, 112 aa, synthetic construct
DIVLTQSPASLAVSLGQRATISCRASESVDSYGNSFMHWYQQKPGQPPKLLIYRASNLESGIPARFSGSGSRTDFTLTINPVEADDVATYYCQQSNEDPLTFGAGTKLELKR

SEQ ID NO: 17 - protein, 117 aa, synthetic construct
QVQLVQSGAEVKKPGASVKVSCKASGYTFSSYWIEWVRQAPGQGLEWIGQILPGIGSTNYNEKFKGRVTITADTSTSTVYMELSSLRSEDTAVYYCARGYGNYGDYWGQGTTVTVSS

SEQ ID NO: 18 - protein, 112 aa, synthetic construct
DIVLTQSPASLAVSPGQRATITCRASESVDSYGNSFMHWYQQKPGQPPKLLIYRASNLESGVPARFSGSGSGTDFTLTINPVEAQDTANYYCQQSNEDPLTFGAGTKLELKR

SEQ ID NO: 19 - DNA, 25 bp, synthetic construct
  source 1..25: mol_type = other DNA
tgtaccgtgtttccttagcttgtgt

SEQ ID NO: 20 - DNA, 25 bp, synthetic construct
  source 1..25: mol_type = other DNA
ggcgagaagggcacccacaagggct

SEQ ID NO: 21 - DNA, 25 bp, synthetic construct
  source 1..25: mol_type = other DNA
agcagctgctgcttagggccgccgg

SEQ ID NO: 22 - DNA, 25 bp, synthetic construct
  source 1..25: mol_type = other DNA
gggcacccacaagggctactgcctg

SEQ ID NO: 23 - DNA, 25 bp, synthetic construct
  source 1..25: mol_type = other DNA
tccccctggagatatgagttggaca

SEQ ID NO: 24 - DNA, 25 bp, synthetic construct
  source 1..25: mol_type = other DNA
gtgtacaaccacttgccatgaaggg

SEQ ID NO: 25 - DNA, 25 bp, synthetic construct
  source 1..25: mol_type = other DNA
cgcaggctgtaccgtgtttccttag

SEQ ID NO: 26 - DNA, 25 bp, synthetic construct
  source 1..25: mol_type = other DNA
gcccatttctggaggccaccactcc

SEQ ID NO: 27 - DNA, 25 bp, synthetic construct
  source 1..25: mol_type = other DNA
gggctactgcctggagcgcaggctg

SEQ ID NO: 28 - DNA, 25 bp, synthetic construct
  source 1..25: mol_type = other DNA
ccaggtgtacaaccacttgccatga

SEQ ID NO: 29 - DNA, 25 bp, synthetic construct
  source 1..25: mol_type = other DNA
ccggggcaactcggagctgctctac

SEQ ID NO: 30 - DNA, 25 bp, synthetic construct
  source 1..25: mol_type = other DNA
ctgtaccgtgtttccttagcttgtg

SEQ ID NO: 31 - DNA, 25 bp, synthetic construct
  source 1..25: mol_type = other DNA
atgccatggcgagaagggcacccac

SEQ ID NO: 32 - DNA, 25 bp, synthetic construct
  source 1..25: mol_type = other DNA
atgccatggcgagaagggcacccac

SEQ ID NO: 33 - DNA, 25 bp, synthetic construct
  source 1..25: mol_type = other DNA
cagctgctgcttagggccgccggaa

SEQ ID NO: 34 - DNA, 25 bp, synthetic construct
  source 1..25: mol_type = other DNA
gcacccacaagggctactgcctgga

SEQ ID NO: 35 - DNA, 25 bp, synthetic construct
  source 1..25: mol_type = other DNA
cccctggagatatgagttggacaga

SEQ ID NO: 36 - DNA, 25 bp, synthetic construct
  source 1..25: mol_type = other DNA
gtacaaccacttgccatgaagggcc

SEQ ID NO: 37 - DNA, 25 bp, synthetic construct
  source 1..25: mol_type = other DNA
gctgtaccgtgtttccttagcttgt

SEQ ID NO: 38 - DNA, 25 bp, synthetic construct
  source 1..25: mol_type = other DNA
aaagttctgcccatttctggaggcc

SEQ ID NO: 39 - DNA, 25 bp, synthetic construct
  source 1..25: mol_type = other DNA
ggctactgcctggagcgcaggctgt

SEQ ID NO: 40 - DNA, 25 bp, synthetic construct
  source 1..25: mol_type = other DNA
caggtgtacaaccacttgccatgaa

SEQ ID NO: 41 - DNA, 25 bp, synthetic construct
  source 1..25: mol_type = other DNA
ccggggcaactcggagctgctctac

SEQ ID NO: 42 - DNA, 26 bp, synthetic construct
  source 1..26: mol_type = other DNA
ctagtgtagttactagtcttttgaca

SEQ ID NO: 43 - DNA, 25 bp, synthetic construct
  source 1..25: mol_type = other DNA
atttgtttgtttactcatcactcag

SEQ ID NO: 44 - DNA, 22 bp, synthetic construct
  source 1..22: mol_type = other DNA
gtaaataattgtgcttcgtcag

SEQ ID NO: 45 - DNA, 20 bp, synthetic construct
  source 1..20: mol_type = other DNA
tagaaaggtgcacgggtgtg

SEQ ID NO: 46 - DNA, 21 bp, synthetic construct
  source 1..21: mol_type = other DNA
caaatctaggcattaacagtg